SEQUENCE LISTING (27 sequences)

SEQ ID NO: 1 - protein, 579 aa, Homo sapiens
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 2 - protein, 13 aa, Artificial Sequence
  Synthetic construct pSer 472 RelB immunogenic peptide
  MOD_RES (4)..(4): PHOSPHORYLATION
GTVSLPGLEPPGG

SEQ ID NO: 3 - protein, 558 aa, Mus musculus
MPSRRAARESAPELGALGSSDLSSLSLTVSRTTDELEIIDEYIKENGFGLDGTQLSEMPRLVPRGPASLSSVTLGPAAPPPPATPSWSCTLGRLVSPGPCPRPYLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGHLHRMDPILSEPVYDKKSTNTSELRICRINKESGPCTGGEELYLLCDKVQKEDISVVFSTASWEGRADFSQADVHRQIAIVFKTPPYEDLEISEPVTVNVFLQRLTDGVCSEPLPFTYLPRDHDSYGVDKKRKRGLPDVLGELSSSDPHGIESKRRKKKPVFLDHFLPGHSSGLFLPPSALQPADSDFFPASISLPGLEPPGGPDLLDDGFAYDPSAPTLFTMLDLLPPAPPLASAVVGSGGAGATVVESSGPEPLSLDSFAAPGPGDVGTASLVGSNMFPNQYREAAFGGGLLSPGPEAT

SEQ ID NO: 4 - protein, 607 aa, Rattus norvegicus
MMPRAPCVQPGLRPYPCAASPAGDRPNRLGCPAARLALVRARPELAFGLSVPTGRAMPSRRAARESAPELGALGSSDIPSLSRTTEIIDEYIKENGFGLDGAQLSEMPRLVPRGPASLSSVTLGPAAPPPPATPPWNCTLGRLVSPGPCPRPYLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGHLHRMDPILSEPVYDKKSTNTSELRICRINKESGPCTGGEELYLLCDKVQKEDISVVFSTASWEGRADFSQADVHRQIAIVFKTPPYEDLEISEPVTVNVFLQRLTDGVCSEPLPFTYLPRDHDSYGVDKKRKRGLPDVLGELSSSDPHGIESKRRKKKPVFLDHFLPGHSSGLFLPPSALQSADTDFFPGTISLPGLEPPGGPDLLDDGFAYDPSAPTLFTMLDLLPPAPPLASAVVGNGGAGATVVESPGPEPLSLDSFAAPGPGDVGTASLVGSNMFPNQYREAAFGGGLLSPGPEAT

SEQ ID NO: 5 - protein, 560 aa, Canis lupus familiaris
MPSRRVARPSAAPELGALGSTDLSSLAVSRTTEIIDEYIKENGFGLEGAQLGPGEGLPRLVSRGAASLSTVTLGPPAPPPPATPPPWGCHLGRVMPQAPGPGPRPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRRKKPAFLDHFLPNSSSGPFLPPSALLPDTDFFPGTVSLPGLEPPGGPDLLDDSFAYDDSTAPTLFTMLDMLPPAPPLASAVTGNGAAGPAVGEPSGPEPMTLDSYQAPGPGDGGTASLVGSNMFPNQYREVGFGGGLLSPGPEAT

SEQ ID NO: 6 - protein, 15 aa, Artificial Sequence
  Synthetic construct L-CDR1 of anti pSer 472 RelB monoclonal antibody
RASKSVYTSGYSYMH

SEQ ID NO: 7 - protein, 7 aa, Artificial Sequence
  Synthetic construct L-CDR2 of anti pSer 472 RelB monoclonal antibody
YASNLQS

SEQ ID NO: 8 - protein, 9 aa, Artificial Sequence
  Synthetic construct L-CDR3 of anti pSer 472 RelB monoclonal antibody
QHNWEIPLT

SEQ ID NO: 9 - protein, 5 aa, Artificial Sequence
  Synthetic construct H-CDR1 of anti pSer 472 RelB monoclonal antibody
TDAMN

SEQ ID NO: 10 - protein, 19 aa, Artificial Sequence
  Synthetic construct H-CDR2 of anti pSer 472 RelB monoclonal antibody
RMRSKSNNYATYYADSVKD

SEQ ID NO: 11 - protein, 8 aa, Artificial Sequence
  Synthetic construct H-CDR3 of anti pSer 472 RelB monoclonal antibody
VYGGAMDY

SEQ ID NO: 12 - protein, 117 aa, Artificial Sequence
  Synthetic construct VH of anti pSer 472 RelB monoclonal antibody
EVQLVETGGGLVQPKGSLKLSCAVSGFTFNTDAMNWVRQAPGKGLEWVARMRSKSNNYATYYADSVKDRCTISRDDSQSMFYLQMNNLKTEDTAMYYCVYGGAMDYWGQGTSVTVSS

SEQ ID NO: 13 - protein, 111 aa, Artificial Sequence
  Synthetic construct VL of anti pSer 472 RelB monoclonal antibody
DIVLTQSPASLVVSLGQRATISCRASKSVYTSGYSYMHWFQQKPGQPPKLLIKYASNLQSGVPARFSGSGSGTDFTLNIHPVEAEDTATYYCQHNWEIPLTFGAGTKLELK

SEQ ID NO: 14 - DNA, 333 bp, Artificial Sequence
  Synthetic construct DNA sequence of VL of anti pSer472 RelB monoclonal antibody
gacattgtgctgacacagtctcctgcttccttagttgtatctctgggacagagggccaccatctcatgcagggccagcaaaagtgtctatacatctggctatagttatatgcactggttccaacagaaaccaggacagccacccaaactcctcatcaagtatgcctccaacctacaatctggggtccctgccaggttcagtggcagtgggtctgggacagacttcaccctcaacatccatcctgtggaggcggaggatactgcaacatattactgtcagcacaattgggagattccgctcacgttcggtgctgggaccaagctggagctgaaa

SEQ ID NO: 15 - DNA, 351 bp, Artificial Sequence
  Synthetic construct DNA sequence of VH of anti pSer472 RelB monoclonal antibody
gaggtgcagcttgttgagactggtggaggattggtgcagcctaaagggtcattgaaactctcatgtgcagtctctggattcaccttcaataccgatgccatgaactgggtccgccaggctccaggaaagggtttggaatgggttgctcgcatgagaagtaaaagtaataattatgcaacatattatgccgattcagtgaaagacaggtgcaccatctccagagatgattcacaaagcatgttctatctgcaaatgaacaacttgaaaactgaggacacagccatgtattactgtgtttacggaggtgctatggactactggggtcaaggaacctcagtcaccgtctcctca

SEQ ID NO: 16 - protein, 563 aa, Aotus nancymaae
MLRSGRASGPSVPTGRAMPSRRVARPPAASELGALGSPDLSSLSLAVSRTTGEHPPPFLLTHTCRMGLGGPRGEVSVWGFTEVHFSFPLTGCIPPCRLVPPAPGPGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMCRMDPVLSEPVYDKKSTNTSELRICRINKESGPCTGGEELYLLCDKVQKEDISVVFSTASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAFLDHFLPSHGSGPFLPPSALLPDPDFFSGTVSLPGLEPPGGPDLLDDGFAYDPTASPLLTMLDLLPPAPPHASAVVCSGGAGAVVGEPPGPEPLTLDSYQGPGPGDGGTASLVGSNMFPNQYREAAFGGGLLSPGPEAT

SEQ ID NO: 17 - protein, 541 aa, Rhinopithecus bieti
SPDLSSLSLTVSRSTDELEIIDEYIKENGFGLDGGQSGPGEGLPRLVSRGAASLSTVTLGPVAPPATPPPWGCPLGRLVPPTQGPGLQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHGSGPFLPPSALLPDPDFFSGTVSLPGLEPPGGPDLLDDGFAYDPTAPTLFTMLDLLPPAPPHASAVVCSAGAVVGEPPGPEPLTLDSYQAPGPGDGGTASLVGSNMFPNQYREAAFGGGLLSPGPEAT

SEQ ID NO: 18 - protein, 579 aa, Saimiri boliviensis
  UNSURE (94)..(121): Xaa at positions 94 to 121 may be any naturally occuring amino acid
MLRSGRASGPSVPTGRAMPSRRVARPPAASELGALGSPDLSSLSLAVSRTTDELEIIDEYIKENGFGLDRAQPGPGEGLPRLVSRGAASLSTVXXXXXXXXXXXXXXXXXXXXXXXXXXXXGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQVCRMDPVLSEPVYDKKSTNTSELRICRINKESGPCTGGEELYLLCDKVQKEDISVVFSTASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAFLDHFLPSHGSGPFLPPSALLPDPDFFSGTVSLPGLEPPGGPDLLDDGFAYDPTATPLLTMLDLLPPAPPHASAVVCSGGAGAVVGEPPGPEPLTLDSYQGPGPGDGGTASLVGSNMFPNQYREAAFGGGLLSPGPEAT

SEQ ID NO: 19 - protein, 440 aa, Chlorocebus sabaeus
MRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHGSGPFLPPSALLPDPDFFSGTVSLPGLEPPGGPDLLDDGFAYDPTAPTLFTMLDLLPPAPPHASAVVCSAGAVVGETPGPEPLTLDSYQALGPGDGGTASLVGSNMFPNQYREAAFGGGLLSPGPEAT

SEQ ID NO: 20 - protein, 541 aa, Rhinopithecus roxellana; identical to SEQ ID NO: 17

SEQ ID NO: 21 - protein, 526 aa, Macaca fascicularis
DELEIIDEYIKENGFGLDGGQPGPGEGLPRLVSRGAASLSTVTLGPVAPPATPPPWGCPLGRLVPPAQGPGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHSSGPFLPPSALLPDPDFFSGTVSLPGLEPPGGPDLLDDGFAYDPTAPTLFTMLDLLPPAPPHASAVVCSAGAVVGEPPGPEPLTLDSYQAPGPGDGGTASLVGSNMFPNQYREAAFGGGLLSPGPEAT

SEQ ID NO: 22 - protein, 522 aa, Macaca fascicularis
IIDEYIKENGFGLDGGQPGPGEGLPRLVSRGAASLSTVTLGPVAPPATPPPWGCPLGRLVPPAQGPGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHSSGPFLPPSALLPDPDFFSGTVSLPGLEPPGGPDLLDDGFAYDPTAPTLFTMLDLLPPAPPHASAVVCSAGAVVGEPPGPEPLTLDSYQAPGPGDGGTASLVGSNMFPNQYREAAFGGGLLSPGPEAT

SEQ ID NO: 23 - protein, 548 aa, Macaca fascicularis
  UNSURE (55)..(77): Xaa at positions 55 to 77 may be any naturally occuring amino acid
MLRSGRASGPSVPTGRAMPSRRVARPPAASELGALGSPDLSSLSLAVSRTTDELXXXXXXXXXXXXXXXXXXXXXXXCSLGRLVPPAPGPGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMCRMDPVLSEPVYDKKSTNTSELRICRINKESGPCTGGEELYLLCDKVQKEDISVVFSTASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAFLDHFLPSHGSGPFLPPSALLPDPDFFSGTVSLPGLEPPGGPDLLDDGFAYDPTASPLLTMLDLLPPAPPHASAVVCSGGAGAVVGEPPGPEPLTLDSYQGPGPGDGGTASLVGSNMFPNQYREAAFGGGLLSPGPEAT

SEQ ID NO: 24 - protein, 538 aa, Macaca fascicularis
SPDLSSLSLTVSRSTEIIDEYIKENGFGLDGGQSGPGEGLPRLVSRGAASLSTVTLGPVAPPATPPPWGCPLGRLVPPTQGPGLQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHGSGPFLPPSALLPDPDFFSGTVSLPGLEPPGGPDLLDDGFAYDPTAPTLFTMLDLLPPAPPHASAVVCSAGAVVGEPPGPEPLTLDSYQAPGPGDGGTASLVGSNMFPNQYREAAFGGGLLSPGPEAT

SEQ ID NO: 25 - protein, 576 aa, Macaca fascicularis
  UNSURE (91)..(118): Xaa at positions 91 to 118 may be any naturally occurring amino acid
MLRSGRASGPSVPTGRAMPSRRVARPPAASELGALGSPDLSSLSLAVSRTTEIIDEYIKENGFGLDRAQPGPGEGLPRLVSRGAASLSTVXXXXXXXXXXXXXXXXXXXXXXXXXXXXGPQPHLVITEQPKQRGMRFRYECEGRSAGSILGESSTEASKTLPAIELRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQVCRMDPVLSEPVYDKKSTNTSELRICRINKESGPCTGGEELYLLCDKVQKEDISVVFSTASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAFLDHFLPSHGSGPFLPPSALLPDPDFFSGTVSLPGLEPPGGPDLLDDGFAYDPTATPLLTMLDLLPPAPPHASAVVCSGGAGAVVGEPPGPEPLTLDSYQGPGPGDGGTASLVGSNMFPNQYREAAFGGGLLSPGPEAT

SEQ ID NO: 26 - protein, 538 aa, Rhinopithecus roxellana; identical to SEQ ID NO: 24

SEQ ID NO: 27 - protein, 419 aa, Macaca fascicularis
ELQRALLFLQLRDCGGLREVEVTACLVWKDWPHRVHPHSLVGKDCTDGVCRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPSHSSGPFLPPSALLPDPDFFSGTVSLPGLEPPGGPDLLDDGFAYDPTAPTLFTMLDLLPPAPPHASAVVCSAGAVVGEPPGPEPLTLDSYQAPGPGDGGTASLVGSNMFPNQYREAAFGGGLLSPGPEAT

(19) United States
(12) Patent Application Publication
(10) Pub. No.: US 2023/0417753 A1
(43) Pub. Date: Dec. 28, 2023

(54) METHODS AND KITS FOR CLASSIFYING SOLID CANCER-AFFLICTED PATIENTS BASED ON RELB LABELLING

(71) Applicants: UNIVERSITÉ PARIS CITÉ, Paris (FR); INSTITUT NATIONAL DE LA SANTE ET DE LA RECHERCHE MEDICALE, Paris (FR); CLINI HOLDING, Nanterre (FR); INSTITUT CURIE, Paris (FR)

(72) Inventors: Véronique BAUD, Paris (FR); Baptiste ELUARD, Courbevoie (FR)

(21) Appl. No.: 18/036,500

(22) PCT Filed: Nov. 18, 2021
(86) PCT No.: PCT/EP2021/082139

§ 371 Date: May 11, 2023

(30) Foreign Application Priority Data
Nov. 18, 2020 (EP) 20306407.6

(51) Int. Cl. G01N 33/574

(52) U.S. Cl. CPC G01N 33/57415; G01N 33/57496; G01N 2440/14

(57) ABSTRACT

An in vitro method belonging to the field of medicine, more specifically the field of cancer prognostic and therapeutic management, is disclosed. The method is for classifying a subject afflicted with a solid cancer as having a good prognosis or a poor prognosis. The method comprises detecting in a cancer cell sample from the subject phosphorylated RelB protein at the serine residue 472 and/or detecting a RelB homolog phosphorylated at a corresponding serine. A kit for implementing the method also is disclosed.

### FIELD OF THE INVENTION

The invention belongs to the field of medicine, more specifically the field of cancer prognostic and therapeutic management. The invention relates to an in vitro method for classifying a subject afflicted with a solid cancer as having a good prognosis or a poor prognosis, said method comprising detecting in a cancer cell sample from said subject phosphorylated RelB protein at the serine residue 472 and/or detecting RelB homolog phosphorylated at a corresponding serine. The invention also relates to a kit for implementing said method.

### BACKGROUND OF THE INVENTION

Cancer is the second leading cause of death globally and is responsible for an estimated 9.6 million deaths in 2018. Globally, about 1 in 6 deaths is due to cancer (World Health Organization, website: who.int). In Europe, cancer incidence is continuously growing and increased by around 50 percent from 2.1 million to 3.1 million cases between 1995 and 2018 (Hofmacher et al., 2019).

Accordingly, the economic impact of cancer is significant and is continuously increasing. Direct costs of cancer doubled from €52 billion to €103 billion in Europe between 1995 and 2018 (Hofmacher et al., 2019), to which should be added significant economic burden of productivity losses due to premature deaths.

Therefore, prognostic factors are of the outmost importance in cancer from several points of view (Mackillop, 2006)):
Patient: Having simple and a clear picture on how the disease and treatments will evolve is important for personal decision-making, notably in terms of finance, employment, accommodation (care) and even psychological and spiritual path.
Treatment: Prognostic factors can help the clinicians to select appropriate therapy for the patients in such a manner that they will potentially benefit for prevention of recurrence of tumour. Avoiding use of inappropriate or ineffective therapy will save time and life duration expectancy for the patient.
Health policy: At the societal level, skills in prognosis are means optimizing the use of resources allowing more efficient care and therefore, avoid waste money due to inappropriate treatments. Both the use of expensive in hopeless situations or undertreatment have adverse consequences from financial point of view. Also, prognostic information may also be useful in making allocational decisions of healthcare systems.
Medical science: prognostic factors are often used in the design, conduct and analysis of clinical trials and in gaining knowledge of the disease.

Hence, there is a need for identifying new prognostic factors for cancer as surrogate and/or complement of those that are currently used.

NF-κB (nuclear factor kappa-light-chain-enhancer of activated B cells) is a protein complex that controls gene transcription. NF-κB is found in almost all animal cell types and is involved in cellular responses to stimuli such as stress, cytokines, free radicals, ultraviolet irradiation, oxidized LDL, and bacterial or viral antigens. It is now well accepted that the NF-κB activation pathways are involved in inflammatory diseases, cancer development and progression in human solid tumours.

In mammals, the NF-κB transcription factor family is composed of five members, RelA (p65), RelB, cRel (Rel), NF-κB1 (p50 and its precursor p105) and NF-κB2 (p52 and its precursor p100), and forms a collection of various homodimeric and heterodimeric complexes (Baud V & Karin M, 2009). The activity of the NF-κB subunit complexes is regulated by two major pathways. The first one, known as the classical or canonical NF-κB activation pathway, mainly applies to RelA:p50 dimers. The second pathway, the so-called alternative or non-canonical NF-κB signaling pathway, implies in RelB-p52 and RelB-p50 dimers (Shao-Cong Sun, 2017).

Accumulated reports indicate the importance of RelB in either solid or hematologic malignancies.

The inventors surprisingly identified various cancer cell labelling patterns related to phosphorylated RelB at its serine residue 472 (ser 472) which are a valuable prognostic factors for solid cancers.

### SUMMARY OF THE INVENTION

The inventors have surprisingly found that detecting phosphorylated RelB at its ser 472 within cells of a cancer cell sample is associated with prognostic factors of cancer and is further in some instance an independent marker in relation with overall survival, distant free metastasis survival and the risk of occurrence of metastases.

Also, it is an object of this invention to provide an in vitro method for classifying a subject afflicted with a solid cancer as having a good prognosis or a poor prognosis, said method comprising detecting in a cancer cell sample from said subject phosphorylated RelB protein of SEQ ID NO:1 at the serine residue 472 and/or detecting RelB homolog phosphorylated at a corresponding serine.

In a particular embodiment, the detection of phosphorylated RelB protein of SEQ ID NO:1 at the serine residue 472 and/or RelB homolog phosphorylated at a corresponding serine is detected using an antibody, or a fragment thereof, preferably a monoclonal antibody, or a fragment thereof, or an aptamer which are ease of use means in the field of cancer diagnostic and in anatomopathological classification of cancer.

More particularly, it has been discovered that both a cytoplasmic labelling and or a nuclear labelling provide valuable information in that fields. Also, an embodiment of the method of the invention, the step of detecting the phosphorylated RelB protein of SEQ ID NO:1 at the serine residue 472 and/or detecting RelB homolog phosphorylated at a corresponding serine comprises detecting:
A cytoplasmic labelling, and/or
A nuclear labelling,
   in the cells of said cancer cell sample, said labelling corresponding to said phosphorylated RelB and/or phosphorylated RelB homolog.

In a particular embodiment of the method, detecting a cytoplasmic labelling for the phosphorylated RelB protein of SEQ ID NO:1 at the serine residue 472 and/or a RelB homolog phosphorylated at a corresponding serine comprises:
When a diffuse labelling is detected, quantifying the intensity of labelling and/or the percentage of cells that are labelled and/or the percentage of the surface of tissue biopsy sample that is labelled, or
When a discrete labelling with dots is observed, quantifying the size of said dots and/or quantifying their number either in cells or in the whole cancer cell sample and/or the percentage of cells of cancer cell sample that are labelled by these dots and/or the percentage of the surface of cancer cell sample that is labelled by these dots.

In another particular embodiment, in the method of the invention, detecting a nuclear labelling for the phosphorylated RelB protein of SEQ ID NO:1 at the serine residue 472 and/or a RelB homolog phosphorylated at a corresponding serine comprises detecting the presence or absence of said nuclear labelling and/or the percentage of cells of cancer cell sample with said nuclear labelling and/or the percentage of the surface of tissue biopsy sample that is labelled with said nuclear labelling.

Subjects for which a nuclear labelling is detected are found to have a significant lower overall survival, an increased risk of metastasis occurrence and/or a lower distant metastases survival. Accordingly, in an embodiment of the method of the invention, detecting a nuclear labelling for the phosphorylated RelB protein of SEQ ID NO:1 at the serine residue 472 and/or a RelB homolog phosphorylated at a corresponding serine is indicative of a poor prognosis in said subject. In a even more particular embodiment a poor prognosis comprises a significant increase of the risk for the occurrence of metastases for said subject and/or of a significant decrease of overall survival expectancy.

Also, a discrete cytoplasmic labelling for the phosphorylated RelB protein is found indicative of an increased risk of suffering from a triple-negative breast cancer for subject suffering of breast cancer. Accordingly, in a particular embodiment of the method of the invention, detecting a discrete labelling with dots in cytoplasm of cancer cell sample is indicative of an increased risk of suffering from a triple-negative breast cancer for said a subject suffering from breast cancer.

A discrete cytoplasmic labelling is found significantly associated with prognostic factors which are indicative of proliferation propensity and correlatively sensitivity to chemotherapy of said cancer. Also, advantageously, in an embodiment of the method of the invention, detecting a discrete labelling with dots in cytoplasm of cancer cell sample is indicative of an increased risk of suffering from a cancer with a high proliferation potential and/or sensitivity to chemotherapy for said subject.

In a very particular embodiment, in the method of the invention, the step of detecting phosphorylated RelB protein of SEQ ID NO:1 at the serine residue 472 and/or detecting RelB homolog phosphorylated at a corresponding serine is performed using an antibody, a fragment thereof, or a single chain antibody comprising at least one CDR sequence selected from SEQ ID NO:6, SEQ ID NO:7, SEQ ID NO:8 SEQ ID NO:9, SEQ ID NO:10, SEQ ID NO:11 or a function-conservative variant thereof.

In an embodiment, the cancer cell sample used in the methods of the invention is a sample of tumour tissue or of metastasis from said subject.

Though the method of the invention can be implemented at any stage of the disease, it presents a particular advantage when subject has not yet been diagnosed with metastases, as it provides information, amongst others, about the risk of occurrence of metastases, thereby providing valuable help to the practitioner in choosing the more convenient therapeutic strategy depending of the labelling for phosphorylated RelB protein that is detected in cancer cells of the subject. Accordingly, in an embodiment of the method of the invention, the subject is free of metastasis.

The method is useful in providing information and allowing classification of subject suffering from any solid cancer, in a particular embodiment, said subject is suffering from a lung cancer, breast cancer, prostate cancer, cervical cancer, pancreatic cancer, colon cancer, ovarian cancer; stomach cancer, oesophagus cancer, mouth cancer, tongue cancer, gum cancer, skin cancer, melanoma, basal cell carcinoma, Kaposi's sarcoma, muscle cancer, heart cancer, liver cancer, bronchial cancer, cartilage cancer, bone cancer, testis cancer, kidney cancer, endometrium cancer, uterus cancer, bladder cancer, spleen cancer, thymus cancer, thyroid cancer, brain cancer, neuron cancer, mesothelioma, gall bladder cancer, ocular cancer, cancer of the cornea, cancer of uvea, cancer of the choroids, cancer of the macula, vitreous humor cancer, joint cancer, a synovium cancer, or a glioblastoma, preferably said subject is suffering from a breast cancer.

In a preferred embodiment, said subject is a mammal, preferably a human.

Another object of the invention is also a kit comprising:
An antibody, or a fragment thereof, preferably a monoclonal antibody, or a fragment thereof, which binds the phosphorylated RelB protein of SEQ ID NO:1 at the serine residue 472 and/or a RelB homolog phosphorylated at a corresponding serine.
Instruction to allow classification of the subject afflicted with cancer as having good or bad prognosis by detecting nuclear and/or cytoplasmic labelling with said antibody or fragment thereof.

### LEGEND OF DRAWINGS

FIG. 1. Nuclear labelling for p5472-RelB correlates with distant metastasis and poor prognosis. Kaplan—Meier estimate of (A) overall survival, (B) distant metastasis-free survival and (C) cumulative incidence of metastases, according to nuclear pSer 472 RelB status as evaluated by tissue microarray based immunohistochemistry in 210 tumour tissues samples. P-values were calculated using a Log-rank (Mantel-Cox) test.

FIG. 2. Example of an immunohistochemical nuclear staining for pSer 472 RelB of a triple negative typed breast cancer from series of breast cancer tumour collection from patients who received a surgery at Institut Curie between 2005 and 2006 (PICBIM series). “A” indicates nuclear staining. Tumour cells also exhibit a diffuse cytoplasmic staining (“B”, intensity score of 2) and discrete cytoplasmic dots (“C”, size score of 1).

FIG. 3. Example of an immunohistochemical diffuse and discrete cytoplasmic staining for pSer 472 RelB of a triple negative, grade III B, breast cancer tumour from series of breast cancer tumour collection from PICBIM series. “A” indicates dots (size score of 2); a diffuse cytoplasmic staining (intensity score of 2 is indicated by “B”) is observed. No nuclear staining is detected.

FIG. 4. Example of an immunohistochemical diffuse and discrete cytoplasmic staining for pSer 472 RelB of a luminal A typed breast cancer tumour from series of breast cancer tumour collection from PICBIM series. “A” indicates dots (size score of 2); a score of 3 is attributed to the diffuse cytoplasmic staining (indicated by “B”). No nuclear staining is detected.

FIG. 5. Example of an immunohistochemical nuclear labelling for pSer 472 RelB of a luminal A typed breast cancer from series of breast cancer tumour collection from patients from PICBIM series. “A” indicate nuclear labelling. Tumour exhibit also diffuse cytoplasmic labelling (“B”, intensity score of 1) and discrete cytoplasmic dots (“C” size score of 1).

### DETAILED DESCRIPTION OF THE INVENTION

### Definitions

As intended herein, the term “comprising” has the meaning of “including” or “containing”, which means that when an object “comprises” one or several elements, other elements than those mentioned may also be included in the object. In contrast, when an object is said to “consist of” one or several elements, the object cannot include other elements than those mentioned.

“Solid cancer” or “solid cancer tumour” refers to any carcinoma or sarcoma, for example carcinoma or sarcoma such as lung cancer, breast cancer, prostate cancer, cervical cancer, pancreatic cancer, colon cancer, ovarian cancer; stomach cancer, oesophagus cancer, mouth cancer, tongue cancer, gum cancer, skin cancer (e.g., melanoma, basal cell carcinoma, Kaposi's sarcoma, etc.), muscle cancer, heart cancer, liver cancer, bronchial cancer, cartilage cancer, bone cancer, testis cancer, kidney cancer, endometrium cancer, uterus cancer, bladder cancer, spleen cancer, thymus cancer, thyroid cancer, brain cancer, neuron cancer, mesothelioma, gall bladder cancer, ocular cancer (e.g., cancer of the cornea, cancer of uvea, cancer of the choroids, cancer of the macula, vitreous humor cancer, etc.), joint cancer (such as synovium cancer), glioblastoma. More preferred cancer to implement method of the invention are cancers to breast cancers, lung cancers, prostate cancers, colorectal cancers, or bone sarcomas, soft tissue sarcomas. In a particularly preferred embodiment “solid cancer” is a breast cancer.

The term “subject” is meant to refer to any mammal, e.g. mouse, rat, monkey, dog, human. In a preferred embodiment the subject is a human subject.

“Cancer cell sample” includes any sample which comprises cancer cells from which the subject to be classified is suffering from. In a particular embodiment, “cancer cell sample” is a sample of tumour tissue, i.e. sample from a biopsy or of tumour resection, or even the tumour tissue itself. In another embodiment, said cancer cell sample is a sample from a metastasis, resulting from either a biopsy or resection. In a particular embodiment said sample is duly prepared to be subjected to common immunohistochemistry (IHC). Except when stated otherwise, “Cancer cell sample” or “cancer sample” have the same signification as defined above and are used interchangeably.

The terms “bad prognosis”, when related to a subject, in the context of the present invention, means that subject is suffering from a solid cancer which is particularly aggressive and/or resistant to current therapies thereby resulting in a significant lowering of overall survival of said subjects, when compared to subjects suffering from a less aggressive solid cancer. Subject of “bad prognosis” is meant to include subjects with worse Overall Survival (OS, defined as the period of time for which patient is alive after disease diagnostic), subjects with worse Progression Free Survival (PFS, the length of time during and after the treatment of the disease, that said subject lives with the disease but it does not get worse), subjects with worse Failure Free Survival (FFS, defined as the period of time with the absence of relapse, non-relapse mortality or addition of another systemic therapy), subjects with worse Event Free Survival (EFS, the length of time after primary treatment for a cancer ends that the patient remains free of certain complications or events that the treatment was intended to prevent or delay) or subjects with worse Distant Metastasis-Free Survival (DMFS, the period of time to the appearance of distant metastasis, e.g., spread of the original tumour to organs or lymph nodes distant thereof). More particularly, subject of “bad prognosis” includes subject with an increased risk of occurrence of metastases a risk for a reduced FFS or DMFS) and/or a risk for a worse OS.

Conversely, subject with a “good prognosis” include subjects for which OS expectancy, PFS, FFS, and/or EFS are in line with or even better than overall statistics in the art, depending on cancer type.

“RelB” is a protein which, in humans, is encoded by the RELB gene (also known as I-REL, IREL, REL-B, RELB proto-oncogene, IMD53); Human RelB protein sequence is accessible under the Uniprot number Q01201 (NCBI Reference Sequence: NP_006500.2). RelB is conserved through the mammal species and numerous homologs of the human RelB protein of SEQ ID NO:1 exist. Amino acids sequence of human RelB (SEQ ID NO:1) is:

(SEQ ID NO: 1)
“Met Leu Arg Ser Gly Pro Ala Ser Gly Pro Ser Val Pro Thr Gly Arg Ala Met Pro Ser Arg Arg
Val Ala Arg Pro Pro Ala Ala Pro Glu Leu Gly Ala Leu Gly Ser Pro Asp Leu Ser Ser Leu Ser
Leu Ala Val Ser Arg Ser Thr Asp Glu Leu Glu Ile Ile Asp Glu Tyr Ile Lys Glu Asn Gly Phe Gly
Leu Asp Gly Gly Gln Pro Gly Pro Gly Glu Gly Leu Pro Arg Leu Val Ser Arg Gly Ala Ala Ser
Leu Ser Thr Val Thr Leu Gly Pro Val Ala Pro Pro Ala Thr Pro Pro Pro Trp Gly Cys Pro Leu Gly
Arg Leu Val Ser Pro Ala Pro Gly Pro Gly Pro Gln Pro His Leu Val Ile Thr Glu GIn Pro Lys Gln
Arg Gly Met Arg Phe Arg Tyr Glu Cys Glu Gly Arg Ser Ala Gly Ser Ile Leu Gly Glu Ser Ser Thr
Glu Ala Ser Lys Thr Leu Pro Ala Ile Glu Leu Arg Asp Cys Gly Gly Leu Arg Glu Val Glu Val Thr
Ala Cys Leu Val Trp Lys Asp Trp Pro His Arg Val His Pro His Ser Leu Val Gly Lys Asp Cys
Thr Asp Gly Ile Cys Arg Val Arg Leu Arg Pro His Val Ser Pro Arg His Ser Phe Asn Asn Leu
Gly Ile GIn Cys Val Arg Lys Lys Glu Ile Glu Ala Ala Ile Glu Arg Lys Ile Gln Leu Gly Ile Asp Pro
Tyr Asn Ala Gly Ser Leu Lys Asn His Gln Glu Val Asp Met Asn Val Val Arg Ile Cys Phe Gln
Ala Ser Tyr Arg Asp Gln Gln Gly Gln Met Arg Arg Met Asp Pro Val Leu Ser Glu Pro Val Tyr
Asp Lys Lys Ser Thr Asn Thr Ser Glu Leu Arg Ile Cys Arg Ile Asn Lys Glu Ser Gly Pro Cys Thr
Gly Gly Glu Glu Leu Tyr Leu Leu Cys Asp Lys Val Gln Lys Glu Asp Ile Ser Val Val Phe Ser
Arg Ala Ser Trp Glu Gly Arg Ala Asp Phe Ser GIn Ala Asp Val His Arg Gln Ile Ala Ile Val Phe
Lys Thr Pro Pro Tyr Glu Asp Leu Glu Ile Val Glu Pro Val Thr Val Asn Val Phe Leu Gln Arg Leu
Thr Asp Gly Val Cys Ser Glu Pro Leu Pro Phe Thr Tyr Leu Pro Arg Asp His Asp Ser Tyr Gly
Val Asp Lys Lys Arg Lys Arg Gly Met Pro Asp Val Leu Gly Glu Leu Asn Ser Ser Asp Pro His
Gly Ile Glu Ser Lys Arg Arg Lys Lys Lys Pro Ala Ile Leu Asp His Phe Leu Pro Asn His Gly Ser
Gly Pro Phe Leu Pro Pro Ser Ala Leu Leu Pro Asp Pro Asp Phe Phe Ser Gly Thr Val Ser Leu
Pro Gly Leu Glu Pro Pro Gly Gly Pro Asp Leu Leu Asp Asp Gly Phe Ala Tyr Asp Pro Thr Ala
Pro Thr Leu Phe Thr Met Leu Asp Leu Leu Pro Pro Ala Pro Pro His Ala Ser Ala Val Val Cys
Ser Gly Gly Ala Gly Ala Val Val Gly Glu Thr Pro Gly Pro Glu Pro Leu Thr Leu Asp Ser Tyr Gln
Ala Pro Gly Pro Gly Asp Gly Gly Thr Ala Ser Leu Val Gly Ser Asn Met Phe Pro Asn His Tyr
Arg Glu Ala Ala Phe Gly Gly Gly Leu Leu Ser Pro Gly Pro Glu Ala Thr”.

Several phosphorylation sites have been already characterized on the RelB protein (Baud V & Collares D, 2016). For example phosphorylation at serine 368 has been shown to be required for NF-κB DNA binding activity, dimerization with other NF-κB subunits (p105/p50, p100/p52), and p100 half-life (Maier H.J. et al., 2003). Of note, no biological function has been associated to said phosphorylation and no inducer has been identified. Phosphorylation of threonine 84 and the serine 552 were found to be associated with the induction of RelB degradation by the proteasome in T cell lines (Marienfeld R. et al., 2001). Phosphorylation on serine 472 was shown to induce dissociation of RelB from its interaction with the inhibitory protein IκBα and to allow its binding to the promoter of critical migration-associated genes (Authier H. et al., 2014).

A “RelB homolog” is a protein whose sequence shares at least 80% homology with the RelB protein of SEQ ID NO:1, while retaining the RelB function, e.g. the capacity of binding p50 or with DNA, that can be demonstrated for example by electrophoretic mobility shift assay (Derruder et al., 2003). Preferably, the amino acid sequences of the homologs of the RelB protein are identical at more than 80%, preferably 81%, more preferably 82%, more preferably 83%, more preferably 84%, more preferably 85%, preferably 86%, more preferably 87%, more preferably 88%, more preferably 89%, more preferably 90%, more preferably 91%, more preferably 92%, more preferably 93%, more preferably 94%, more preferably 95%, more preferably 96% to the and even more preferably 97% to SEQ ID NO:1. Preferably, amino acid sequence identity is measured by using the global alignment algorithm of Needleman and Wunsch (1970). It is noteworthy that the amino acid sequence of RelB is highly conserved among the mammalian species but serine 472 may correspond to a slightly different position in the said homolog amino acid sequence.

Consequently, as used herein, the terms “corresponding serine in a RelB homolog” or “corresponding serine” refer to the serine residue in a RelB homolog which corresponds to the serine 472 of SEQ ID NO:1 when said RelB homolog is aligned with SEQ ID NO:1 with a conventional software. For example, serine 451 is the corresponding serine residue in RelB homolog of the species Mus musculus (NCBI Reference Sequence: NP_033072.2).

Throughout this text “phosphorylated RelB protein at the serine residue 472” or “phosphorylated Ser 472 RelB” or “pSer 472 RelB” can be used interchangeably to designates either phosphorylated RelB protein of SEQ ID NO:1 at the serine residue 472 or a RelB homolog phosphorylated at a corresponding serine, except when otherwise specified.

### Methods of the Invention

Method of Classifying Subjects Afflicted with a Solid Cancer

The inventors have been able to identify and select specific patterns of phosphorylated Ser 472 RelB protein labelling on cancer cells that are associated with poor prognosis for subject from which cancer cells have been taken. More particularly, these patterns are found associated with poor OS and/or increased likelihood of relapse, i.e. increased likelihood of cancer spreading and metastasis occurrence in said subjects.

Accordingly in a first aspect, the present invention relates to an in vitro method for classifying a subject afflicted with a solid cancer as having a good prognosis or a poor prognosis, comprising detecting in a cancer cell sample from said individual phosphorylated RelB protein of SEQ ID NO:1 at the serine residue 472 and/or detecting RelB homolog phosphorylated at a corresponding serine.

In an embodiment, in the in vitro method for classifying a subject afflicted with a solid cancer of the invention, detecting the phosphorylated RelB protein of SEQ ID NO:1 at the serine residue 472 and/or detecting RelB homolog phosphorylated at a corresponding serine comprises detecting:
a cytoplasmic labelling, and/or
a nuclear labelling,
   in the cells of said solid cancer cell sample, said labelling corresponding to said phosphorylated RelB and/or phosphorylated RelB homolog.

The cytoplasmic and/or nuclear labelling pattern of phosphorylated Ser 472 RelB in cancer cell sample can be characterized by several features, for example, by either its aspect (e.g. discrete or diffuse), the subcellular localization of the label, the intensity of labelled areas, the percentage of cells that are labelled, or even as the percentage of the surface of the cancer cell sample that is specifically labelled for phosphorylated Ser 472 RelB.

Also, in a particular embodiment, the in vitro method of the invention comprises, when a diffuse cytoplasmic labelling of phosphorylated Ser 472 RelB is detected, a step of quantifying the intensity of labelling and/or the percentage of the surface of solid cancer cell sample that is labelled.

In another particular embodiment, the in vitro method of the invention comprises, when a discrete cytoplasmic labelling of phosphorylated Ser 472 RelB with dots is detected, a step of quantifying the size of said dots and/or quantifying their number either in cells or in the whole cancer cell sample and/or quantifying the percentage of cells of cancer cell sample that are labelled by these dots and/or the percentage of the surface of the solid cancer cell sample that is labelled by these dots. For instance, number of dots in cancer cell sample can be determined as a mean number of dots based upon the number of cells in the cancer cell sample or as a mean number of dots in positively labelled cells with this discrete cytoplasmic labelling of phosphorylated Ser 472 RelB. In a preferred embodiment, number of dots in cancer cell sample is determined as a mean number of dots in positively labelled cells with this discrete cytoplasmic labelling of phosphorylated Ser 472 RelB.

In a further embodiment, the in vitro method of the invention comprises a step of characterizing the nuclear labelling of cells in solid cancer cell sample that is labelled. In this regard, in a more particular embodiment, said method comprises detecting the presence or absence of said nuclear labelling and/or the percentage of the surface of tissue biopsy sample that is labelled and/or the number of cells (i.e. as a percentage of total number of cells) that are labelled with said nuclear labelling of phosphorylated Ser 472 RelB.

Further to the characterization and quantifying steps of phosphorylated Ser 472 RelB as exposed above, the in vitro method can comprise a step of scoring of cancer cell sample based upon the above-mentioned labelling characterization and quantification items, which are considered independently or combined to obtain a global score.

An example of a scoring system is provided in tables 1, 2 and 3 below.

TABLE 1
Diffuse cytoplasmic labelling items

Labelling Intensity = B

| % of positive | Labelling | |
|---|---|---|
| cells = A % | Intensity | score |
| ]0%-10%[ | No labelling | 0 |
| [10%-50%[ | Mild labelling | 1 |
| [50-80%[ | Moderate | 2 |
| | labelling | |
| ≥80% | Intense labelling | 3 |

Final score Diffuse cytoplasmic labelling = A × B

TABLE 2
Discrete (dots) cytoplasmic labelling items

| Size of dots = | % of positive | |
|---|---|---|
| A Size | cells = B | |
| 0 | ]0%-10%[ | Number of dots |
| 1 | [10%-50%[ | per labelled |
| 2 | [50-80%[ | cells = C |
| | ≥80% | |

Final score discrete cytoplasmic labelling = A × B × C

TABLE 3
Nuclear labelling items

| Presence of nuclear | |
|---|---|
| labelling | % of positive |

| labelling | Score | cells = B % |
|---|---|---|
| Yes | 1 | ]0%-10%[ |
| | | [10%-50%[ |
| No | 0 | [50-80%[ |
| | | ≥80% |

Final score nuclear labelling = A × B

The inventors have found that identifying a nuclear labelling for phosphorylated Ser 472 RelB in a cancer cell sample from a subject is indicative of a poor prognosis for said subject. More particularly a subject, a cancer cell sample of which is found positive for a nuclear labelling for phosphorylated Ser 472 RelB, presents a greater incidence of metastasis formation. In other words, said subject has a greater risk to develop metastasis, even after tumour resection, in comparison to the ones cancer cell sample of which does not present said nuclear labelling. Also, it has also been found that patients cancer cell sample of which was found positive for nuclear labelling for phosphorylated Ser 472 RelB showed a significant shorter DMFS. Said subjects can be thus classified as subjects having a PFS, EFS, DM FS or FFS significantly shorter than other subjects that do not present such labelling. Further subjects showing positive nuclear labelling for Ser 472 RelB in a cancer cell sample have also been found to display a significant lower OS than subjects for whom a cancer cell sample is classified as negative for said nuclear labelling for phosphorylated Ser 472 RelB.

Accordingly, in an embodiment, the invention relates to a method for classifying a subject afflicted with a solid cancer as having a good prognosis or a poor prognosis, wherein detecting a nuclear labelling, in a cancer cell sample from said subject, for phosphorylated RelB protein of SEQ ID NO:1 at the serine residue 472 and/or detecting RelB homolog phosphorylated at a corresponding serine, is indicative of a poor prognosis in said subject.

In a particular embodiment, in said method, a cancer cell sample will be found positive as soon as one cell showing a nuclear labelling for phosphorylated RelB protein of SEQ ID NO:1 at the serine residue 472 and/or for a RelB homolog phosphorylated at a corresponding serine will be identified in said cancer cell sample. Accordingly, in this embodiment, the subject from which said cancer cell sample comes from is classified as subject as of a poor prognosis in relation with its cancer disease. In a more particular embodiment, said subject is classified as a subject with an increased risk of occurrence of metastases. In another particular embodiment, said subject is classified as at risk for a shorter DMFS than with subjects for whom no such a labelling is detected. In another particular embodiment, said subject is classified as at risk for a reduced OS in comparison with subjects for whom no such a labelling is detected.

In a more particular embodiment, a cancer cell sample will be found positive for a nuclear labelling for phosphorylated RelB protein of SEQ ID NO:1 at the serine residue 472 and/or for a RelB homolog phosphorylated at a corresponding serine when at least 10%, more preferably at least 50% even more preferably at least 80% of cells from cancer cell sample will be found with a nuclear labelling for phosphorylated RelB protein at the serine residue 472, as determined for example in table 3. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In another particular embodiment, said subject is classified as at risk for a shorter DMFS than with subjects for whom no such a labelling is detected. In another particular embodiment, said subject is classified as at risk for a reduced OS in comparison with subjects for whom no such a labelling is detected.

Method of Prognosing/Diagnosing Cancer Comprising pSer 472 RelB Labelling

The inventors have also found that the presence of a cytoplasmic labelling for phosphorylated RelB protein at the serine residue 472 is of use in prognosing and/or diagnosing cancer.

For example, a discrete labelling, under the form of dots that are observed throughout the cytoplasm of some cells of cancer cell sample, for phosphorylated RelB protein at the serine residue 472 is found to be significantly associated with prognostic factors currently used in cancer diagnosis and prognosis such as mitotic index, Ki-67 index, presence of tumour emboli. Also, a diffuse cytoplasmic labelling is also found to significantly correlate with known prognostic or diagnostic factors of cancer; though to a less extent, especially with TI Ls, mitotic and Ki-67 indexes.

The mitotic index is a well-known means to measure cellular proliferation. It is defined as the percentage of cells undergoing mitosis in a given population of cells. An elevated mitotic index indicates more cells are dividing. Determination of mitotic index implies counting the number of mitoses within the tumour sample, from a tissue section prepared and stained according to conventional techniques, at a high magnification. Though easy to determine, it necessitates a sufficiently large sample available to be analyzed, which is not always the case. The mitotic index is currently used as a factor predicting both overall survival and response to chemotherapy. Cancer with a high mitotic index are supposed to be more sensitive to chemotherapy than cancers in which cells are dividing at a low rate, but have as a corollary, a great propensity to expand.

Ki-67 index determination is also a way to measure cellular proliferation potential of cancer cells. Ki-67 is a nuclear protein which is expressed in all phases of the cell cycle except during the GO phase. In other, words, Ki-67 is present in the nucleus of proliferative cells, in phase G1, S, G2 and M. The Ki-67 labelling index represents the percentage of nuclei stained by the Ki-67 antibody within a cell sample using a Ki-67 specific antibody detected using usual immunohistochemistry and immunofluorescence techniques. Therefore Ki-67 index represents the percentage of cells that have entered cell division cycle, which does not mean that they will effectively divide.

Generally, both the Ki-67 and the mitotic indexes are used to determine cellular proliferation potential in cancer prognosis and/or diagnosis and to make a choice between treatment options, which necessitates the implementation of two different histological methods. Detection of a cytoplasmic labelling for phosphorylated RelB protein at the serine residue 472 in cancer cell sample therefore also represents an advantageous surrogate to the use of mitotic index and Ki-67 for determining proliferation potential and sensitivity to chemotherapy of cancer.

The presence of tumour emboli corresponds to the presence of clusters of tumour cells in the vessels. Obviously, tumour emboli are only detected in case of anatomical analysis of whole resected tumour. Presence of tumour emboli means the tumour has access to the circulation, and therefore risks being already disseminated. Presence of emboli is therefore an important prognostic factor for cancer which also determine the treatment options for the afflicted subject.

Therefore, an object of the invention is the use of the above-mentioned labelling features for phosphorylated RelB protein at the serine residue 472 found in cells from cancer cell sample in the aim of cancer diagnosis or prognosis. More particularly, in some instance at least one of these labelling features, preferably a combination of them, can be used as an item of anatomopathological examination of cancer cell sample or even as a surrogate for some of items currently used in the tumour grading and/or cancer diagnosis and/or cancer prognosis, but which are, e.g. less convenient to determine. Common items used in tumour grading or cancer prognosis are, in a non-limitative way, tumour grade, tumour stage, Ki-67 index, mitotic index, emboli, tumor-infiltrating lymphocytes quantification (TILs).

As exemplified in table 2, in case of a discrete cytoplasmic labelling, either the mean number of dots per labelled cells, their size, or the number of cells that are labelled (as a percentage of labelled cells in cancer cell sample) can be considered independently or to determine a final score of discrete cytoplasmic labelling, said final score being for example the result of the product of each of number of dots, size score, and percentage of labelled cells (table 2).

Also in an embodiment, a cancer cell sample will be found positive for a discrete cytoplasmic labelling for phosphorylated RelB protein of SEQ ID NO:1 at the serine residue 472 and/or for a RelB homolog phosphorylated at a corresponding serine when a score (as determined in table 2) of at least 1, more preferably at least 2 for the size of dots will be determined in cells from cancer cell. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In another particular embodiment, said subject is classified as at risk for a lower OS.

In a particular embodiment, a score of 1 in regard with the dot size criterion will be attributed when the dots in the solid cancer cell sample labelled for pSer 472 RelB are only visible if using a magnification above 10×, a score of 2 when they are visible using a magnification as small as 10×.

In another embodiment, a cancer cell sample will be found positive for a discrete cytoplasmic labelling for phosphorylated RelB protein of SEQ ID NO:1 at the serine residue 472 and/or for a RelB homolog phosphorylated at a corresponding serine when (as determined in table 2) more than 0% but less than 10% of cells, more preferably from 10 to less than 50% of cells, even more preferably at least 50% of cells in term of the percentage of labelled cells in the cancer cell sample will be determined. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In other words, said subject is classified as with an increased risk of a lower PFS, DMFS, FFS and or EFS. In another particular embodiment, said subject is classified as at risk for a lower OS.

In a particular embodiment, a final score for a discrete cytoplasmic labelling of cancer cell is determined for the cancer cell sample of the subject, level of which is indicative, for the subject from which said cancer cell sample comes from, of a poor prognosis in relation with its cancer disease; said final score can be based on any (combination of) features used to measure or rank said discrete labelling. In a particular embodiment said final score for a discrete labelling is based on at least one of item selected from number of dots per cell, size of dots, percentage of cells that are labelled in the sample, or combination thereof. In an even more particular embodiment, said final score can be defined as specified in table 2, i.e. as the product of each items (number of dots per cell, size of dots, percentage of cells that are labelled in the sample). In a more particular embodiment, the level of said final score is indicative of the risk of occurrence of metastases. In other words, said subject is classified as with an increased risk of a lower PFS, DMFS, FFS and or EFS. In another particular embodiment, the level of said final score is indicative of the risk for a lower OS in the subject.

In another embodiment, a cancer cell sample will be found positive for a discrete cytoplasmic labelling for phosphorylated RelB protein of SEQ ID NO:1 at the serine residue 472 and/or for a RelB homolog phosphorylated at a corresponding serine when of at least 10%, more preferably at least 20% even more preferably at least 30% of cells of the cancer cell sample will be detected. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In other words, said subject is classified as with an increased risk of a lower PFS, DMFS, FFS and or EFS. In another particular embodiment, said subject is classified as at risk for a lower OS.

As exemplified in table 1, in case of a diffuse cytoplasmic labelling, either intensity of the labelling, or the number of cells that are labelled (as a percentage of labelled cells in cancer cell sample) can be considered independently or to determine a final score of diffuse cytoplasmic labelling, which corresponds to the product of the percentage of labelled cells with the intensity level of cytoplasmic labelling. Intensity of labelling is a typical criterion used by skilled in the art in the field of histopathology.

Also in an embodiment, a cancer cell sample will be found positive for a diffuse cytoplasmic labelling for phosphorylated RelB protein of SEQ ID NO:1 at the serine residue 472 and/or for a RelB homolog phosphorylated at a corresponding serine when a score (as determined in table 1) of at least 1, more preferably at least 2, even more preferably at least 3 for the intensity of said cytoplasmic labelling will be determined in cells from cancer cell. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In other words, said subject is classified as with an increased risk of a lower PFS, DMFS, FFS and or EFS. In another particular embodiment, said subject is classified as at risk for a lower OS.

In another embodiment, a cancer cell sample will be found positive for a diffuse cytoplasmic labelling for phosphorylated RelB protein of SEQ ID NO:1 at the serine residue 472 and/or fora RelB homolog phosphorylated at a corresponding serine when (as determined in table 1) more than 0% but less than 10% of cells, more preferably from 10% to less than 50% of cells, even more preferably at least 50% of cells in term of the percentage of cells with a diffuse cytoplasmic pSer 472 RelB labelling in the cancer cell sample will be determined in cells from cancer cell. Accordingly, in this particular embodiment, the subject from which said cancer cell sample comes from is classified as subject as of a poor prognosis in relation with its cancer disease. In a more particular embodiment, said subject is classified as a subject with an increased risk of occurrence of metastases. In other words, said subject is classified as with an increased risk of a lower PFS, DMFS, FFS and or EFS. In another particular embodiment, said subject is classified as at risk for a lower OS.

In a particular embodiment, a final score for a diffuse cytoplasmic labelling of cancer cell is determined for the cancer cell sample of the subject, level of which is indicative, for the subject from which said cancer cell sample comes from, of a poor prognosis in relation with its cancer disease; said final score can be based on any (combination of) features used to measure or rank said discrete labelling. In a particular embodiment said final score for a diffuse labelling is be defined as specified in table 1, i.e. as the product of intensity of labelling and percentage of cells that are labelled in the cancer cell sample. In a particular embodiment, the level of said final score is indicative of the risk of occurrence of metastases. In other words, said subject is classified as with an increased risk of a lower PFS, DMFS, FFS and or EFS. In another particular embodiment, the level of said final score is indicative of the risk for a lower OS in the subject.

Of note final score as specified in table 1 is found to be significantly associated with factors of bad prognosis in cancer (experimental section).

It should be contemplated that, as shown in the experimental section, the occurrence of nuclear, diffuse and/or discrete cytoplasmic labelling within a cancer cell sample are not mutually exclusive. In other words, within the same cancer cell sample, as exemplified in the experimental section, the three types of labelling can be observed and analyzed.

Though effective in classifying a subject afflicted with any solid cancer, the subject to be classified through the method of the invention is preferably a subject afflicted with breast cancer. Also in a particular embodiment, the method of the invention in any of its embodiments as described above is applied to a breast cancer cell sample from a subject suffering from breast cancer. In a more particular embodiment said subject afflicted with breast cancer is a human subject.

In this regard, another particular embodiment of the method of the invention relates to an in vitro method for classifying a human subject afflicted with a breast cancer as having a good prognosis or a poor prognosis said method comprising detecting a nuclear labelling for the phosphorylated RelB protein of SEQ ID NO:1 at the serine residue 472 and/or a RelB homolog phosphorylated at a corresponding serine, wherein said labelling is indicative of a poor prognosis in said human subject.

Also, another particular embodiment of the method of the invention relates to an in vitro method for classifying a human subject afflicted with a breast cancer as having a good prognosis or a poor prognosis said method comprising detecting a diffuse and/or a discrete cytoplasmic labelling, as exposed above.

In an even more particular embodiment, the in vitro method for classifying human afflicted with breast cancer of the invention is applied in conjunction with search for other well-known prognostic/risk factors of breast cancer as the stage of the disease (TNM), tumour grade, presence of emboli, ki-67 index, mitotic index, tumour-infiltrating lymphocytes (TILs) assessment, expression of estrogen receptor (ER), progesterone receptor (PR), HER2 receptor.

Molecular characterization of breast cancer as Triple Negative Breast Cancer is statistically associated with discrete cytoplasmic labelling of cancer cell in regard with the phosphorylated RelB protein of SEQ ID NO:1 at the serine residue 472 and/or a RelB homolog phosphorylated at a corresponding serine. Also, said labelling pattern constitute a valuable surrogate marker to help in identification of TNBC, especially when the assignment as TNBC is dubious when using conventional methods.

Phosphorylated RelB protein at the serine residue 472 detection and labelling means

In a preferred embodiment, the phosphorylation of the RelB protein on serine 472 or on the corresponding serine of a RelB homolog, is detected by means of an antibody or of an aptamer.

Said antibody or aptamer, is specific of the RelB protein phosphorylated on serine 472 and as such will not detect other phosphorylated serine in other proteins.

### Antibody

In a preferred embodiment, said antibody is an isolated phosphorylation site-specific antibody that specifically binds the RelB protein of SEQ ID NO:1 or a homolog thereof only when said protein or homolog is phosphorylated on serine 472 or on a corresponding serine. Preferably, said antibody does not bind said RelB protein or homolog when it is not phosphorylated on said serine.

The terms “antibody”, “antibodies” or “immunoglobulin” are used interchangeably throughout this application. They should be construed in the broadest sense: these terms, as used herein, thus include monoclonal antibodies (e.g., full length or intact monoclonal antibodies), polyclonal antibodies, multivalent antibodies or multispecific antibodies (e.g., bispecific antibodies so long as they exhibit the desired biological activity) and functional fragments thereof.

The term “antibody” as used herein designates a polypeptide that exhibits binding specificity to a specific antigen. More particularly, an antibody (or “immunoglobulin”) consists of a glycoprotein comprising at least two heavy (H) chains and two light (L) chains interconnected by disulfide bonds.

Each heavy chain comprises a heavy chain variable region (or domain) (abbreviated herein as V_{H}) and a heavy chain constant region (hereafter C_{H}). Heavy chains are classified as gamma, mu, alpha, delta, or epsilon, and define the antibody's isotype as IgG, IgM, IgA, IgD, and IgE, respectively. The C_{H} region of the immunoglobulin IgG, IgD, and IgA (γ, δ and α chains respectively) comprises three domains (CH1, CH2, and CH3) and a hinge region for added flexibility, while the C_{H} region of the immunoglobulin IgM and IgE contains 4 domains (CH1, CH2, CH3, and CH4).

IgG antibodies are classified in four distinct subtypes, named IgG1, IgG2, IgG3 and IgG4. The structure of the hinge regions in the γ chain gives each of these subtypes its unique biological profile (even though there is about 95% similarity between their Fc regions, the structure of the hinge regions is relatively different).

Each light chain comprises a light chain variable region (abbreviated herein as V_{L}) and a light chain constant region comprising only one domain, C_{L}. There are two types of light chain in mammals: the kappa (κ) chain, encoded by the immunoglobulin kappa locus on chromosome 2, and the lambda (λ) chain, encoded by the immunoglobulin lambda locus on chromosome 22.

The V_{H} and V_{L} regions can be further subdivided into regions of hypervariability, termed “Complementarity Determining Regions” (CDR), which are primarily responsible for binding an antigen, and which are interspersed with regions that are more conserved, designated “Framework Regions” (FR). Each V_{H} and V L is composed of three CDRs and four FRs, arranged from amino-terminus to carboxy-terminus in the following order: FR1, CDR1, FR2, CDR2, FR3, CDR3, FR4. The assignment of amino acid sequences to each domain is in accordance with well-known conventions (see e.g. Lefranc, M.-P., et al., (2003)). The functional ability of the antibody to bind a particular antigen depends on the variable regions of each light/heavy chain pair, and is largely determined by the CDRs. The variable region of the heavy chain differs in antibodies produced by different B cells, but is the same for all antibodies produced by a single B cell or B cell clone (or hybridome). By contrast, the constant regions of the antibodies mediate the binding of the immunoglobulin to host tissues or factors, including various cells of the immune system (e.g. effector cells) and the first component (C1q) of the classical complement system.

“Antibody” as used herein also designates “single chain antibody” or sdAb, or related molecules, which refers to the single heavy chain variable domain (VHH) of antibodies of the type that can be found in camelids which are naturally devoid of light chains. Such single domain antibodies are also named nanobodies. They represent the smallest antibody fragments (around 14 kDa) able to preserve the binding affinity and specificity of the original whole antibody and they are appreciated for their structural stability and their simple engineering into reagents suitable for in vitro and in vivo applications (de Marco (2020)). They can be obtained by classical immunization of animals known to produce such antibodies, e.g. camelids or cartilaginous fishes with the desired antigen and subsequent isolation of the mRNA coding for heavy-chain antibodies. They can be also produced and purified using genetic engineering as reviewed in de Marco (2020).

As used herein, the term “antibody fragment” intends to designate Fab, Fab′, F(ab′)2, scFv, dsFv, ds-scFv, dimers, minibodies, diabodies, multimers thereof or bispecific antibody fragments. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, dimers, minibodies, diabodies, bispecific antibody fragments and other fragments can also be synthesized by recombinant techniques. For example, scFvs, comprise the variable regions of the immunoglobulin heavy and light chain, covalently connected by a peptide linker. These “antibody fragments” are rather small in comparison with antibodies and generally retain specificity and affinity for antigen in a single polypeptide and can provide a convenient building block for larger, antigen-specific molecules.

“Antibody” as used herein also designates any combination of part(s) or of fragment(s) of antibodies as described above provided that it retains sufficient affinity constant and/or dissociation constant for phosphorylated RelB protein, as defined below. For example, both scFv fragments or VHH nanobodies can be linked to the Fc fragment of the desired species and keep their specificity and binding properties.

An “epitope” is the site on the antigen to which an antibody binds. It can be formed by contiguous residues or by non-contiguous residues brought into close proximity by the folding of an antigenic protein. In particular, an epitope can comprise a residue carrying a specific post-translational modification, e.g. a glycosylation or a phosphorylation, said specific post-translational modification ensuring specific reconnaissance by the antibody. For example, in the present case, the epitope which is recognized by the antibody is a group of contiguous residues or by non-contiguous residues brought into close proximity by the folding of an antigenic protein, said residues comprising the phosphorylated serine 472 residue or a corresponding serine residue. By contrast, the same group of residues comprising the unphosphorylated serine 472 is not recognized by the antibody to be used in the method of the invention.

A “functional fragment” of an antibody means, in particular, an antibody fragment as defined above, with the same binding activity to phosphorylated RelB as the parental antibody.

In the context of the present invention, an antibody or fragment thereof is said to “recognize” or “bind” a peptide having a define sequence if said antibody has an affinity constant Kₐ (which is the inverted dissociation constant, i.e. 1/K_{d}) higher than 10⁶ M⁻¹, preferably higher than 10⁷ M⁻¹, more preferably higher than 10⁸ M⁻¹ for said peptide. Also, in the context of the present invention, an antibody is said to “specifically bind” or to “specifically recognize” a peptide if said antibody or fragment thereof has an affinity constant K a greater than 10⁷ M⁻¹, preferably greater than 10⁸ M⁻¹, more preferably greater than 10⁹ M⁻¹ for said peptide and even more preferably greater than 10¹⁰ M⁻¹ for said peptide and has an affinity constant K a lower than 10⁵ M⁻¹ for all the other peptide.

The affinity constant which is used to characterize the binding of antibodies (Ab) to a peptide or an antigen (Ag) is the inverted dissociation constant defined as follows:

$$Ab + Ag ⇌ AbAg$$

$${K}_{a} = \frac{[ AbAg ]}{[ Ab ] [ Ag ]} = \frac{1}{{K}_{d}}$$

This affinity can be measured for example by equilibrium dialysis or by fluorescence quenching, both technologies being routinely used in the art.

In a preferred embodiment, the antibody to be used in method of the invention binds the Ser 472 phosphorylated RelB protein with a K_{d} of less than 10⁻⁷ M, preferably from less than 10⁻⁸ M. In a further preferred embodiment, the antibodies to be used in method of the invention bind the Ser 472 phosphorylated RelB protein with a K_{d} of less than 10⁻⁹ M, preferably from less than 10⁻¹⁰ M.

More preferably, the antibodies to be used in the methods of the invention do not bind the RelB protein when said protein is not phosphorylated on Ser 472 (or on a corresponding serine residue). In particular, the antibodies of the method of the invention have an affinity constant K a which is less than 10⁵ M⁻¹ for the RelB protein which is not phosphorylated on Ser 472. More particularly, the antibodies to be used in methods of the invention have an affinity constant Kₐ which is less than 10⁵ M⁻¹ for all polypeptides, except with the Ser 472-phosphorylated RelB protein.

The antibody of the methods the invention may be monoclonal or polyclonal and may be of any species of origin, including (for example) mouse, rat, rabbit, horse, or human, or may be a chimeric antibody.

A “polyclonal antibody” as used herein, refers to an antibody that is obtained from different B cells. It typically includes various antibodies directed against various determinants, or epitopes, of the target antigen. Polyclonal phosphorylation site specific antibodies that specifically bind RelB only when phosphorylated at the serine 472 or a corresponding serine residue may be produced by standard antibody production methods, for example by i) immunizing a suitable animal (e.g., rabbit, goat, etc.) with the phosphorylated protein of the invention or with an immunogenic peptide, ii) collecting immune serum from the animal, and iii) separating the polyclonal antibodies from the immune serum, in accordance with known procedures.

Immunogenic peptides suitable for producing antibodies against pSer 472 RelB protein, are constructed and employed according to techniques used in the art (see e.g. Czernik, Methods In Enzymology, 1991; Merrifield, J. Am. Chem. Soc. 1962). Preferably, said immunogenic peptide comprises only a portion of the protein of SEQ ID NO. 1 or of a homolog thereof immediately flanking the phosphorylatable serine 472. In other words, the immunogenic peptide is a peptide of a specific length, said peptide comprising a group of residues which necessarily includes serine 472.

Preferred immunogenic peptides are peptides consisting essentially of about 10 to 15 amino acids of SEQ ID NO:1 including the phosphorylated serine 472 or a corresponding serine in a RelB homolog, wherein about 3 to 8 amino acids are positioned on each side of said phosphorylated serine.

For example, the immunogenic peptide of SEQ ID NO:2 may be used to produce the antibodies suitable to be used in method or kits of the invention. It will be appreciated by those of skill in the art that longer or shorter immunogenic peptides may also be employed. In a preferred embodiment, the immunogenic peptide used to produce antibodies against pSer 472 RelB has the sequence GTVSLPGLEPPGG (SEQ ID NO:2), the Serine in position 4 of said SEQ ID NO:2 being phosphorylated (PO₃H₂).

This immunogenic peptide can be synthetized by conventional means and can be used to generate a polyclonal antibody suitable to be used in the method of the invention.

In a preferred embodiment, the said phosphorylation site-specific antibody is a monoclonal antibody, even more preferably from IgG isotype.

A “monoclonal antibody”, as used herein, means an antibody arising from a nearly homogeneous antibody population. The individual antibodies of a population are identical except for a few possible naturally-occurring mutations which can be found in minimal proportions. In other words, a monoclonal antibody consists of a homogeneous antibody arising from the growth of a single cell clone (for example a hybridoma, a eukaryotic host cell transfected with a DNA molecule coding for the homogeneous antibody, a prokaryotic host cell transfected with a DNA molecule coding for the homogeneous antibody, etc.) and is characterized by heavy chains of one and only one isotype and subtype, and light chains of only one type. Monoclonal antibodies are highly specific and are directed against a single epitope of an antigen. Monoclonal antibodies may be produced by a single clone of B cells or “hybridoma”. Monoclonal antibodies may also be recombinant, i.e. produced by protein engineering. The invention relates to monoclonal antibodies isolated or obtained by purification from natural sources or obtained by genetic recombination or chemical synthesis.

The monoclonal antibodies suitable for the method of the invention may be produced in a hybridoma cell line according to the well-known technique of Kohler and Milstein, 1975; Kohler and Milstein, 1976; see also, CURRENT PROTOCOLS IN MOLECULAR BIOLOGY, Ausubel et al. (1989). Monoclonal antibodies are preferably used in the method of classifying subject afflicted with solid cancer of the invention.

For example, a solution containing the appropriate antigen may be injected into a mouse or other species and, after a sufficient time (in keeping with conventional techniques), the animal is sacrificed, and spleen cells obtained. The spleen cells are then immortalized by fusing them with myeloma cells, typically in the presence of polyethylene glycol, to produce hybridoma cells. The hybridoma cells are then grown in a suitable selection media, such as hypoxanthine-aminopterin-thymidine (HAT), and the supernatant screened for monoclonal antibodies having the desired specificity, as described below. The secreted antibody may be recovered from tissue culture supernatant by conventional methods such as precipitation, ion exchange or affinity chromatography, or the like.

Monoclonal Fab fragments may also be produced in Escherichia coli by recombinant techniques known to those skilled in the art (W. Huse, 1989; Mullinax et al., 1990). If monoclonal antibodies of one isotype are preferred for a particular application, particular isotypes can be prepared directly, by selecting from the initial fusion, or prepared secondarily, from a parental hybridoma secreting a monoclonal antibody of different isotype (Steplewski, et al., 1985; Spira et al., 1984). Recombinant cells for producing an antibody suitable to be used in the method of the invention cells may be constructed by well-known techniques; for example the antigen combining site of the monoclonal antibody can be cloned by PCR and single-chain antibodies produced as phage-displayed recombinant antibodies or soluble antibodies in E. coli.

Antibodies (or fragment thereof) to be used in the methods and kits of the invention specifically bind the RelB protein when phosphorylated on serine 472 (or a corresponding serine in a RelB homolog) and do not bind to the non-phosphorylated form. This specificity may be screened according to standard techniques (Czernik et al., 1991) such as ELISA. Also, peptide competition assays may be carried out to confirm lack of reactivity with other epitopes on the RelB protein. The antibodies may also be tested by Western blotting against cell preparations containing RelB proteins mutated on the serine 472 residue, so as either to not accept phosphorylation (e.g. serine to alanine mutants, Authier et al., 2014) or to mimic constitutive phosphorylation on this residue (e.g. serine to aspartate or glutamate mutants). Such mutations are well known in the art. Antibodies may be further characterized via immunohistochemical (IHC) staining using normal and pathologic tissues to examine RelB-Ser 472 phosphorylation and activation status of the NF-κB pathway in said tissues. IHC may be carried out on paraffin-embedded tissues according to well-known techniques, for example comprising the steps of: i) deparaffinizing tissue sections with xylene followed by ethanol; ii) hydrating in water then PBS; iii) unmasking antigen by heating slide in sodium citrate buffer; iv) incubating sections in hydrogen peroxide; v) blocking in blocking solution; vi) incubating slide in primary antibody and secondary antibody; and finally vii) detecting using ABC avidin/biotin method according to manufacturer's instructions (see ANTIBODIES: A LABORATORY MANUAL, Chapter 10, Harlow & Lane Eds., Cold Spring Harbor Laboratory (1988)). The antibodies may be further characterized by flow cytometry carried out according to standard methods (Chow et al., 2001).

Antibodies (or antibody fragment) specific to Ser 472 phosphorylated RelB (SEQ ID NO:1) or RelB homolog phosphorylated at a corresponding serine can be detected in immunohistochemistry using numerous means well known form the skilled in the art. For example, they can be advantageously conjugated to fluorescent dyes (e.g. Alexa-488, Phycoerythrin (PE), Fluorescein isothiocyanate (FITC)) for use in multiparametric analyses along with other signal transduction and/or cell marker antibodies. Said antibodies can also be biotinylated to be further revealed using horseradish coupled streptavidin or other equivalent means. Otherwise said antibodies can also be detected by an indirect method principle of which is well known from the skilled in the art; briefly in the indirect labelling IHC methods, antibodies specific to Ser 472 phosphorylated RelB (SEQ ID NO:1) or RelB homolog phosphorylated at a corresponding serine is used as primary antibody and is not conjugated is allowed to react with cancer cell sample, and a labeled secondary antibody directed against isotype of the animal species in which the primary antibody has been raised which conjugated either with a fluorescent dye or with an enzyme (such as, e.g., peroxidase, alkaline phosphatase or glucose oxidase) or a molecular prey (such a biotin) for enzymatic detection. The indirect method is known to be more sensitive because of signal amplification resulting from the binding of several secondary antibodies on the primary antibody. In a preferred embodiment, in the method of the invention of detecting phosphorylated RelB protein of SEQ ID NO:1 at the serine residue 472 and/or detecting RelB homolog phosphorylated at a corresponding serine in a cancer cell sample of a subject afflicted with a solid cancer, in any of the above embodiments, is implemented by using antibody(ies) specific to Ser 472 phosphorylated RelB protein in immunohistochemical (IHC) staining of said cancer cell sample, which is a currently used method in the field of diagnosis and reviewed, for example by Ramos-Vara and Miller (2014). Of course, the phosphorylation-site specific antibody to be used in the methods of the invention specifically binds homolog RelB proteins that are phosphorylated at the serine 472 site or a corresponding serine thereof, for example RelB homologs from other species (e.g. mouse SEQ ID NO:3, rat SEQ ID NO:4, dog SEQ ID NO:5, monkey SEQ ID NO: 16 to 27).

In a preferred embodiment, in the method of the invention, the subject afflicted by a solid cancer is a human subject. Also, in this embodiment, the antibody (or fragment thereof) to be used for detecting pSer 472 RelB, in the cancer cell sample from said human subject, specifically binds human phosphorylated RelB protein at serine 472; in other words, as exposed above it does not bind to RelB protein not phosphorylated at serine 472. In a preferred embodiment, said antibody is the mouse monoclonal antibody produced by clone RA3-AF3. Antibodies produced from that clone are particularly suitable to be used in the methods of the invention (experimental section).

Clone RA3-AF3 has been deposited at Institut Pasteur on 18 Nov. 2020 and is available from the Institut Pasteur (CNCM, 25 rue du Docteur Roux, F-75724 PARIS Cedex 15, France) under deposit No. CNCM 1-5612.

In this regard, in an embodiment, in the method of the invention, the antibody (or fragment thereof) to be used for detecting pSer 472 RelB, in a solid cancer cell sample from a subject, is a monoclonal antibody that specifically binds phosphorylated RelB protein at serine 472 and comprises a light chain wherein the variable domain comprises at least one CDR having a sequence selected from the group consisting of SEQ ID NO:6 for L-CDR1, SEQ ID NO:7 for L-CDR2, or SEQ ID NO:8 for L-CDR3 as listed in table 4 below.

In another embodiment the antibody (or fragment thereof) to be used for detecting pSer 472 RelB, in a solid cancer cell sample from a subject, is a monoclonal antibody that specifically binds phosphorylated RelB protein at serine 472 and comprises a heavy chain wherein the variable domain comprises at least one CDR having a sequence selected from the group consisting of SEQ ID NO:9 for H-CDR1, SEQ ID NO:10 for H-CDR2, or SEQ ID NO:11 for HCDR3 as listed in table 4 below.

TABLE 4

| anti-pSer 472 | |
|---|---|
| RelB antibody | |
| IgG Domains | Protein Sequence |
| VH | EVQLVETGGGLVQPKGSLKLSCAVSGFTFNTDAMNWVRQAPG |
| | KGLEWVARMRSKSNNYATYYADSVKDRCTISRDDSQSMFYLQ |
| | MNNLKTEDTAMYYCVYGGAMDYWGQGTSVTVSS (SEQ ID |
| | NO: 12) |
| H CDR1 | TDAMN (SEQ ID NO: 9) |
| H CDR2 | RMRSKSNNYATYYADSVKD (SEQ ID NO: 10) |
| H CDR3 | VYGGAMDY (SEQ ID NO: 11) |
| VL | DIVLTQSPASLVVSLGQRATISCRASKSVYTSGYSYMHWFQQK |
| | PGQPPKLLIKYASNLQSGVPARFSGSGSGTDFTLNIHPVEAEDT |
| | ATYYCQHNWEIPLTFGAGTKLELK (SEQ ID NO: 13) |
| L CDR1 | RASKSVYTSGYSYMH (SEQ ID NO: 6) |
| L CDR2 | YASNLQS (SEQ ID NO: 7) |
| L CDR3 | QHNWEIPLT (SEQ ID NO: 8) |

Table 4. Protein sequence coding the variable domain of the light and heavy chain (V_{L} and V_{H}) of one IgG useful in the methods of the invention.

TABLE 5

| anti-pSer 472 | |
|---|---|
| RelB antibody | |
| IgG Domains | Nucleotide Sequence |
| VL | GACATTGTGCTGACACAGTCTCCTGCTTCCTTAGTTGTATCTCT |
| | GGGACAGAGGGCCACCATCTCATGCAGGGCCAGCAAAAGTGT |
| | CTATACATCTGGCTATAGTTATATGCACTGGTTCCAACAGAAAC |
| | CAGGACAGCCACCCAAACTCCTCATCAAGTATGCCTCCAACCT |
| | ACAATCTGGGGTCCCTGCCAGGTTCAGTGGCAGTGGGTCTGG |
| | GACAGACTTCACCCTCAACATCCATCCTGTGGAGGCGGAGGA |
| | TACTGCAACATATTACTGTCAGCACAATTGGGAGATTCCGCTC |
| | ACGTTCGGTGCTGGGACCAAGCTGGAGCTGAAA (SEQ ID |
| | NO: 14) |
| VH | GAGGTGCAGCTTGTTGAGACTGGTGGAGGATTGGTGCAGCCT |
| | AAAGGGTCATTGAAACTCTCATGTGCAGTCTCTGGATTCACCT |
| | TCAATACCGATGCCATGAACTGGGTCCGCCAGGCTCCAGGAA |
| | AGGGTTTGGAATGGGTTGCTCGCATGAGAAGTAAAAGTAATAA |
| | TTATGCAACATATTATGCCGATTCAGTGAAAGACAGGTGCACC |
| | ATCTCCAGAGATGATTCACAAAGCATGTTCTATCTGCAAATGA |
| | ACAACTTGAAAACTGAGGACACAGCCATGTATTACTGTGTTTA |
| | CGGAGGTGCTATGGACTACTGGGGTCAAGGAACCTCAGTCAC |
| | CGTCTCCTCA (SEQ ID NO: 15) |

Table 5. Nucleotide sequence coding the variable domain of the light and heavy chain (V_{L} and V_{H}) of one IgG useful in the methods of the invention.

In a more particular embodiment, said antibody (or fragment thereof) to be used for detecting pSer 472 RelB comprises a light chain wherein the variable domain comprises at least one CDR having a sequence selected from the group consisting of SEQ ID NO:6 for L-CDR1, SEQ ID NO:7 for L-CDR2 and SEQ ID NO:8 for L-CDR3 and a heavy chain wherein the variable domain comprises at least one CDR having a sequence selected from the group consisting of SEQ ID NO:9 for H-CDR1, SEQ ID NO:10 for H-CDR2 and SEQ ID NO:11 for H-CDR3.

In an even more particular embodiment, in the method of the invention, the antibody (or fragment thereof) to be used for detecting pSer 472 RelB, in a solid cancer cell sample from a subject, is a monoclonal antibody that specifically binds phosphorylated RelB protein at serine 472 comprising:
a heavy chain variable region comprising SEQ ID NO:9 in the H-CDR1 region, SEQ ID NO:10 in the H-CDR2 region and SEQ ID NO:11 in the H-CDR3 region; and
a light chain variable region comprising SEQ ID NO:6 in the L-CDR1 region, SEQ ID NO:7 in the L-CDR2 region and SEQ ID NO:8 in the L-CDR3 region.

Also, in another particular embodiment, the heavy chain variable region (V_{H}) of said antibody has the amino acid sequence set forth as SEQ ID NO:12 and/or the light chain variable region (V_{L}) has the amino acid sequence set forth as SEQ ID NO:13 (table 4).

In another particular embodiment, said antibody, fragment thereof, e.g. a ScFV, or single chain antibody, comprises at least one CDR sequence selected from SEQ ID NO:6, SEQ ID NO:7, SEQ ID NO:8 SEQ ID NO:9, SEQ ID NO:10, SEQ ID NO:11.

In a particular embodiment, in the method of the invention, the antibody (or fragment thereof) to be used for detecting pSer 472 RelB, in a solid cancer cell sample from a subject, is a monoclonal IgG1 comprising sequences as exposed above.

As already mentioned, “Antibody” is meant to encompass single chain antibody as defined above. Also, “antibody fragments” intends to designate Fab, Fab′, F(ab′)2, scFv, dsFv, ds-scFv, dimers, minibodies, diabodies, multimers thereof or bispecific antibody fragments, provided of course it shows a suitable Kₐ or K_{d} for RelB as defined above.

The above specified antibodies are particularly suitable to be used in the method according to the invention, more particularly using IHC methods well known in the art. Also, in a preferred embodiment the method of the invention of detecting phosphorylated RelB protein of SEQ ID NO:1 at the serine residue 472 and/or detecting RelB homolog phosphorylated at a corresponding serine in a cancer cell sample of a subject afflicted with a solid cancer, is implemented by using monoclonal antibody(ies) comprising sequences as exposed above, in an immunohistochemical (IHC) staining of said cancer cell sample.

In an embodiment, said antibody is purified from an hybridoma clone comprising a nucleic acid sequence coding for at least one polypeptide selected from SEQ ID NO:6, SEQ ID NO:7, SEQ ID NO:8, SEQ ID NO:9, SEQ ID NO:10 and SEQ ID NO:11. Also, in a particular embodiment, said hybridoma clone comprises a nucleic acid sequence coding the heavy chain variable region (V_{H}) of said antibody of nucleotide sequence SEQ ID NO:15 and/or a nucleic acid sequence coding for the light chain variable region (V_{L}) of said antibody of nucleotide sequence SEQ ID NO:14 (table 5).

Further to the conventional methods to obtain hybridoma cells producing a monoclonal antibody suitable to implement the method of the invention an example of which is cited above, fragment or derivative of antibodies suitable for the present invention may be produced by any technique known in the art, such as recombinant processes, any chemical, biological, genetic or enzymatic technique of the art, either alone or in combination. Indeed, knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus off the shelf. Also, for example antibodies can be obtained as DNA expression products after incorporation of DNA sequences encoding the antibodies or part thereof as described above into expression vectors and introduction of such vectors into suitable eukaryotic or prokaryotic hosts that will express the desired antibodies, from which they can be later isolated using well-known techniques. Suitable vectors comprise as a plasmid, cosmid, episome, artificial chromosome, phage or a viral vector. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

So, a further object of the invention relates to a vector comprising a nucleic acid of the invention. Such vectors may comprise regulatory elements, such as a promoter, enhancer, terminator and the like, to cause or direct expression of said antibody upon administration to a subject.

Any expression vector for animal cell can be used, so long as a gene encoding the human antibody C region can be inserted and expressed. Examples of suitable vectors include pAGE107 (Miyaji H et al. 1990), pAGE103 (Mizukami T et al. 1987), pHSG274 (Brady G et al. 1984), pKCR (O'Hare K et al. 1981), pSGI beta d2-4-(Miyaji H et al. 1990) and the like. Other examples of plasmids include replicating plasmids comprising an origin of replication, or integrative plasmids, such as for instance pUC, pcDNA, pBR, and the like. Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc.

Protocols for producing such replication-defective recombinant viruses are techniques commonly known in the art.

A further object of the present invention relates to a host cell which has been transfected, infected or transformed by a nucleic acid and/or a vector according to the invention. The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA has been “transformed”. The nucleic acids of the invention may be used to produce an antibody suitable to be used in the method of the invention in a suitable expression system. The term “expression system” means a host cell and compatible vector under suitable conditions, e.g. for the expression of a protein coded for by foreign DNA carried by the vector and introduced to the host cell. Common expression systems include E. coli host cells and plasmid vectors, insect host cells and Baculovirus vectors, and mammalian host cells and vectors.

Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.).

Modifications and changes may be made in the structure of the antibodies of the present invention, and in the DNA sequences encoding them, and still obtain a functional molecule that encodes an antibody with desirable characteristics, i.e. specific to pSER472 RelB with an affinity constant Kₐ greater than 10⁷ M⁻¹, preferably greater than 10⁸ M⁻¹, more preferably greater than 10⁹ M-1 for said peptide and even more preferably greater than 10¹⁰ M⁻¹ for said peptide and has an affinity constant Kₐ lower than 10⁵ M⁻¹ for all the other peptide. In another embodiment, the encoded antibody has a K_{D} of less than 10⁻⁷ M, less than 10⁻⁸ M, less than 10⁻⁹ M, preferably from less than 10⁻¹⁰ M.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

Also, antibodies suitable for the methods of the invention also encompasses function-conservative variants of the antibodies as described above. More particularly function-conservative variants of anyone of the CDR regions as described above.

“Function-conservative variants” are those in which a given amino acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method. A “function-conservative variant” also includes a polypeptide which has at least 60% amino acid identity as determined by BLAST or FASTA algorithms, preferably at least 75%, more preferably at least 80%, more preferably at least 85%, still preferably at least 90%, and even more preferably at least 95%, and which has the same or substantially similar properties or functions as the native or parent protein to which it is compared.

Two amino acid sequences are “substantially homologous” or “substantially similar” when greater than 80%, preferably greater than 85%, preferably greater than 90%, preferably greater than 95%, of the amino acids are identical or similar (functionally identical) over the whole length of the shorter sequence. Preferably, the similar or homologous sequences are identified by alignment using, for example, the GCG (Genetics Computer Group, Program Manual for the GCG Package, Version 7, Madison, Wisconsin) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said antibodies, without appreciable loss of their biological activity, and amongst other Kₐ and K_{d} in regard with pSER472 RelB as mentioned above.

It is known in the art that certain amino acids may be substituted by other amino acids having a similar hydropathic index or score and still result in a protein with similar biological activity, i.e. still obtain a biological functionally equivalent protein. As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well-known to those of skill in the art and include: arginine and lysine; glutamate and aspartate; serine and threonine; glutamine and asparagine; and valine, leucine and isoleucine.

Accordingly, the invention also provides an antibody comprising a heavy and light chain wherein the variable domain comprises:
a H-CDR1 having at least 80% or 85% identity with sequence set forth as SEQ ID NO:9,
a H-CDR2 having at least 80%, at least 85%, preferably at least 90%, more preferably at least 95% identity with sequence set forth as SEQ ID NO:10,
a H-CDR3 having at least 80%, at least 85%, preferably at least 90%, more preferably at least 95% identity with sequence set forth as SEQ ID NO:11,
a L-CDR1 having at least 80%, at least 85%, preferably at least 90%, more preferably at least 95% identity with sequence set forth as SEQ ID NO:6,
a L-CDR2 having at least 80%, at least 85%, preferably at least 90%, more preferably at least 95% identity with sequence set forth as SEQ ID NO:7,
a L-CDR3 having at least 80%, at least 85%, preferably at least 90%, more preferably
at least 95% identity with sequence set forth as SEQ ID NO:8, and that specifically binds to pSER 472 RelB with substantially the same affinity as an antibody comprising a heavy chain wherein the variable domain comprises SEQ ID NO:9 for H-CDR1, SEQ ID NO:10 for H-CDR2 and SEQ ID NO:11 for H-CDR3 and a light chain wherein the variable domain comprises SEQ ID NO:6 for L-CDR1, SEQ ID NO:7 for L-CDR2 and SEQ ID NO:8 for L-CDR3, and more preferably with substantially the same affinity as an anti-pSER 472 RelB antibody with a VL domain of SEQ ID NO: 12 and/or a V_{H} domain of SEQ ID NO: 13.

### Aptamers

In another embodiment, an aptamer is used to detect Ser 472 phosphorylated RelB or RelB homolog phosphorylated at a corresponding serine in the method according to the invention (Neuberger et al., 1984) in the method of the invention. Said aptamer is preferably a nucleic acid-based aptamer (i.e. either RNA or DNA aptamer). Nucleic acid-based aptamers are being developed for a variety of diagnostic applications, including detection of a wide range of non-nucleic acid analytes (Conrad et al., 1996). Aptamers can be selected in vitro by the SELEX process from very large populations of random sequence oligomers (Ellington & Szostak, 1990). This well-established methodology selects aptamers based on their affinity for a specific target molecule. Aptamers can be selected against nearly any class of molecule including proteins, ranging from simple peptides to post-translationally modified proteins. The post-translational modifications potentially detectable by aptamers include a variety of common covalent modifications such as phosphorylation, glycosylation, and proteolytic cleavage and noncovalent modifications such as conformational changes due to binding of ligands (McCauley et al., 2003). Aptamers do not depend on immunological reaction of animals to be produced. Aptamers present the interest of being produced either by chemical synthesis or in bacteria and are thus more quickly and more reproducibly produced and available than antibodies. As antibodies, aptamers can be selected based upon their affinity for the ligand for which they are produced (see e.g. in Ahirwar et al., 2016). Example of production and uses of aptamers in so called aptohistochemistry in cancer are provided e.g. in Ahirwar et al. (2016) or Zamay et al. (2017). Aptamers can be conjugated, as antibodies do, to either fluorescent dyes or other detection means or streptavidin-based amplification techniques.

Method of Adapting or Determining Cancer Treatment Strategy of a Subject Afflicted with a Solid Cancer

Cancer treatment strategy can be inferred from Ser 472 phosphorylated RelB (or RelB homolog at corresponding serine) labelling patterns which are found by the inventors associated with poor OS and/or increased likelihood of relapse, i.e. increased likelihood of cancer spreading and metastasis occurrence in the subject from which sample has been obtained. Moreover, as shown in the experimental section, some of these patterns are found to be associated with commonly used prognostic factors in cancer management and grading.

Accordingly, a further object of the invention relates also to a method of adapting and/or determining cancer treatment strategy of a subject afflicted with a solid cancer, said method comprising identifying specific patterns of phosphorylated Ser 472 RelB protein labelling as exposed above.

In a more particular embodiment, when a diffuse or discrete cytoplasmic phosphorylated Ser 472 RelB protein cytoplasmic labelling is observed in cancer cell sample of the subject, the method further comprises the step of including a chemotherapy in the therapeutic plan for said subject, as those labelling patterns are shown to be associated with high proliferative indexes (Ki-67 index and mitotic index). In an even more preferred embodiment when a discrete phosphorylated Ser 472 RelB protein cytoplasmic labelling is observed in cancer cell sample of the subject, the method further comprises the step of including a chemotherapy in the therapeutic plan for said subject.

It has also been found that a diffuse or discrete cytoplasmic phosphorylated Ser 472 RelB protein cytoplasmic labelling in a cancer cell sample of a subject afflicted by breast cancer is associated with triple negative breast cancer. Triple-negative breast cancer is considered to be more aggressive and have a poorer prognosis than other types of breast cancer, mainly because there are fewer targeted medicines that treat triple-negative breast cancer. Studies have shown that triple-negative breast cancer is more likely to spread beyond the breast and more likely to recur after treatment (website: breastcancer.org/symptoms/diagnosis/trip_neg). Further, triple negative breast cancers are known to be resistant to hormonal therapy or to medicines that target Her2 protein. Hence, in a more particular embodiment, the invention relates to a method of adapting or determining cancer treatment strategy for a subject afflicted with breast cancer, said method comprising, when cancer cell sample of said subject is found positive for a diffuse or discrete cytoplasmic labelling as described above, then not applying to said subject an hormone therapy or anti HER2 therapy to said subject. Also, in another particular embodiment, the invention relates to a method of adapting or determining cancer treatment strategy for a subject afflicted with breast cancer, said method comprising, when cancer cell sample of said subject is found positive for a diffuse or discrete cytoplasmic labelling as described above, then applying chemotherapy or immunotherapy (CAR-T cells or immune checkpoint inhibitors).

As shown in the experimental section, nuclear labelling for phosphorylated Ser 472 RelB protein is the best prognostic factor when compared to other factors as UICC grade, Ki-67 or HER2 status. Indeed, subjects in cancer cell sample of which a nuclear labelling is observed for phosphorylated Ser 472 RelB protein have 3.7 more risk of metastasis occurrence also their DMFS is significantly decreased as well as their OS, in comparison with negative pSer 472 RelB nuclear labelling. This constitute a valuable information in cancer management in these subjects which implies, for example, cancer monitoring exam at a higher frequency than performed for the subject less prone to metastasis occurrence. Also, more aggressive chemotherapy, more extensive surgical operation, and/or other treatment can be considered be used in these subjects in order to more efficiently prevent or delay metastasis occurrence. For example, surgical operation can comprise tumour resection if it has not been done, extended surgical resection which comprises removing the tumour but also tissue next to it, or even removal of the whole organ or another nearby organ wherein otherwise less extensive operation would be considered: for example in case of breast cancer it can encompass not only tumour resection but also total mastectomy instead. Also, in another particular embodiment, the invention relates to a method of adapting or determining cancer treatment strategy for a subject afflicted with breast cancer, said method comprising, when cancer cell sample of said subject is found positive for nuclear labelling as described above, then increasing monitoring exam frequency and/or performing tumour resection or mastectomy and/or applying a chemotherapy treatment prescribed in case of aggressive expanding cancer.

### Kits of the Invention

The invention also relates to a kit suitable to implement the methods of the invention, through the detection of a cytoplasmic or nuclear labelling for Ser 472 phosphorylated RelB protein of cancer cell sample.

Accordingly, a further object of the invention is kit comprising:
an antibody, preferably a monoclonal antibody, or a fragment thereof, or an aptamer specific to the phosphorylated RelB protein of SEQ ID NO:1 at the serine residue 472 and/or detecting RelB homolog phosphorylated at a corresponding serine, and
Instruction to allow classification of the subject afflicted with cancer as having good or bad prognosis by detecting nuclear and/or cytoplasmic labelling with said antibody.

Instructions for the analysis can comprise instruction in relation with how to analyze images resulting from cancer cell labelling with either the aforementioned antibody, fragment thereof or aptamer. In a particular embodiment said instructions can comprise instructions for attributing a score to said solid cancer cell sample based on tables 1, 2 and/or 3.

Also more particular embodiment, the invention relates to a kit comprising:
an antibody, or a fragment thereof, specific to the phosphorylated RelB protein of SEQ ID NO:1 at the serine residue 472 and/or detecting RelB homolog phosphorylated at a corresponding serine,
Instruction to allow classification of the subject afflicted with cancer as having good or bad prognosis by detecting nuclear and/or cytoplasmic labelling with said antibody wherein said instructions comprises attributing a score to said solid cancer cell sample based on, for example, tables 1, 2 and/or 3.

In another particular embodiment, the invention relates to a kit comprising:
an antibody, or a fragment thereof, specific to the phosphorylated RelB protein of SEQ ID NO:1 at the serine residue 472 and/or detecting RelB homolog phosphorylated at a corresponding serine, and
instructions for attributing a score to said solid cancer cell sample based on, for example, tables 1, 2 and/or 3.

In another particular embodiment, the invention relates to a kit comprising:
an antibody, or a fragment thereof, comprising at least one CDR sequence selected from SEQ ID NO:6, SEQ ID NO:7, SEQ ID NO:8, SEQ ID NO:9, SEQ ID NO:10, SEQ ID NO:11 or a function-conservative variant thereof, and
instructions for attributing a score to said solid cancer cell sample based on, for example, tables 1, 2 and/or 3.

In another particular embodiment, the invention relates to a kit comprising:
an antibody, or a fragment thereof, comprising
   a heavy chain variable region comprising SEQ ID NO:9 in the H-CDR1 region, SEQ ID NO:10 in the H-CDR2 region and SEQ ID NO:11 in the H-CDR3 region, or a functional variant hereof; and
   a light chain variable region comprising SEQ ID NO:6 in the L-CDR1 region, SEQ ID NO:7 in the L-CDR2 region and SEQ ID NO:8 in the L-CDR3 region, or a functional variant hereof.
instructions for attributing a score to said solid cancer cell sample based on, for example, tables 1, 2 and/or 3.

In another particular embodiment, the invention relates to a kit comprising:
an antibody, or a fragment thereof, comprising
   a heavy chain variable region comprising SEQ ID NO:12 or a functional variant hereof; and
   a light chain variable region comprising SEQ ID NO:13 or a functional variant hereof.
instructions for attributing a score to said solid cancer cell sample based on, for example, tables 1, 2 and/or 3.

In a more preferred embodiment, the invention relates to a kit comprising:
the mouse monoclonal antibody produced by clone RA3-AF3, or a fragment thereof, and
instructions for attributing a score to said solid cancer cell sample based on, for example, tables 1, 2 and/or 3.

In a more preferred embodiment said kit of the invention is suitable to be used in any IHC staining experiment, and optionally contains further reagents of current use in IHC staining. Accordingly, instructions provided with the kit can also provide directive on how performing IHC on the cancer cell sample of the subject with said antibody or fragment thereof together with instruction to allow classification of the subject afflicted with cancer as having good or bad prognosis by detecting nuclear and/or cytoplasmic labelling with said monoclonal antibody, based on, for example, tables 1, 2 and/or 3.

Further aspects and advantages of the invention will be disclosed in the following examples, which should be considered illustrative.

### EXAMPLE

### Material and Methods

### Cancer Cell Sample

Breast cancer tumour sample were obtained from series of breast tumours from patients who received surgery at Institut Curie (26 rue d′Ulm, 75005 Paris) between 2005 and 2006, named the PICBIM series (from “programme incitatif et collaboratif-Cancer du sein: invasion et motilité”). 234 samples were selected based upon following exclusion criteria:
Subject with metastases upon cancer diagnostic
Subject with bilateral breast cancer
Subject with no medical follow-up.

Distribution of molecular subtypes of breast cancer within these 234 samples is as followed:

TABLE 6

| Molecular subtype | N | % |
|---|---|---|
| Triple negative breast | 68 | 29.1 |
| cancer (TNBC) | | |
| Luminal A | 56 | 23.9 |
| Luminal B | 54 | 23.1 |
| Her2+ | 47 | 20.1 |
| Luminal B/Her2+^{†} | 9 | 3.8 |
| Total | 234 | 100 |

^{†}patients for who it has been possible to classify tumour as of either Luminal B or Her2+ subtype

### Antibody

Mouse Ser 472 phophorylated RelB protein monoclonal antibody produced by the RA3-AF3 hybridoma described above (available at Neo Biotech, #NB-19-00001).

### Immunohistochemistry

Immunohistochemical staining of formalin-fixed paraffin-embedded tumour tissues was performed following conventional automated protocols on BOND III (Leica) automated IHC stainer, using a dilution of the RA3-AF3 antibody of 1/10 000 and the Bond Polymer Refine Detection kit (Leica Biosystems).

Cancer cell pattern for Ser472 phosphorylated RelB

Following features of staining patterns were determined from immunohistochemistry labelling of TMAs of cancer cell sample for the Ser 472 phosphorylated RelB:
Diffuse cytoplasmic labelling: intensity (score from 0 to 3), and % of cells that are labelled
Discrete (dots) cytoplasmic labelling: size of the dot (scored from 0 to 2), number of dots (mean par labelled cells), % of cells that are labelled
Nuclear labelling: presence or not, and % of cells that are labelled

### Statistical Analyses

### Kaplan Meier Estimate

The Kaplan-Meyer estimate of overall survival (OS), distant metastasis-free survival (DMFS) and cumulative incidence of metastasis were performed using log-rank test (Mantel-Cox) on the 210 (out of the 234) breast cancer tumour samples of the cohort for which treatment information of patients were available. Cancer cell samples were censored on the date the patient was last known to be alive or, for patients died as a result of causes unrelated to the breast cancer or treatment, the date of death. Distant metastasis-free survival or overall survival were measured from the date of diagnosis of the disease to that of metastasis detection or death.

Multivariate analysis for Overall Survival, Distant Metastases Free Survival and Cumulative Incidence of Metastasis

The predictive ability of pSer472 RelB staining features of the invention was compared to that of the following prognostic factors: mitotic Index (1-3), KI67>=20%, vascular emboli presence, age (<40 vs >=40), Histological grade (SBR=1 or 2 vs SBR=3), TNBC molecular type (vs others), HER+ amplification status, and UICC grade. Multivariate statistical analyses were performed using Cox proportional hazards model. The predictive ability of each parameter was determined by comparing the hazard ratios. All statistical analyses were performed by using IBM® SPSS® statistics software.

Statistical Association with Prognostic Factors

P values relating pSer472 RelB labelling pattern of selected 234 breast cancer tumour samples to discrete variables were calculated with the use of a Fisher's exact test. Ten prognostic factors were considered: age of the subject upon cancer diagnosis (>40 or <40); TNBC molecular type (vs others); lymph node status (N+ or N−); Size and location of the tumour (T1-2 vs T3-4); UICC grade (1-3); grade 1-3; presence of emboli; mitotic index (1-3); Ki-67 index (0-25%, 25-50%,50-75%, 75-100%); TILs(0-20%, 20-40%,40-60%, 60-80%).

### Results

Nuclear labelling for pS472 RelB is an independent prognostic factor of metastasis occurrence, Overall Survival and Distant Metastasis-Free Survival.

Multivariate analysis of data of the 210 cancer cell samples that have been selected identified factors that are found independent prognostic factors for Cumulative Incidence of Metastasis (table 7), Over Survival (table 8), and Distant metastasis-free survival (table 9) out of which pS472 RelB appears to be the most significative for the three endpoints. The risks of metastases occurrence (table 7), of having a shortened time survival (table 8) or of the occurrence for distant metastases (table 9) are respectively 3.7, 3.8 and 6.4 higher in patients for who a nuclear pSer472 RelB staining is observed vs those subjects for which no such a labelling is observed.

TABLE 7

| | Cumulative incidence of metastases | | |
|---|---|---|---|
| Prognostic factor | HR | 95% C.I. | p-value |
| UICC grade | 2.632 | 2.322-2.941 | 0.002 |
| Ki-67 >=20 | 9.435 | 8.334-10.536 | 0.041 |
| Age < 40 | 0.368 | −0.148-0.884 | 0.053 |
| pSer 472 RelB nuclear labelling | 3.718 | 3.302-4.134 | 0.002 |
| (percentage of cells) | | | |
| HER2 negative | 0.190 | −0.598-0.978 | 0.035 |
| Mitotic index | 0.728 | 0.284-1.172 | 0.475 |
| Vascular emboli | 0.864 | 0.450-1.278 | 0.724 |
| TNBC status | 0.663 | 0.181-1.145 | 0.393 |
| Histological Grade (SBR) = 3 | 3.248 | 2.483-4.012 | 0.123 |

HR: Hazard Ratio

TABLE 8

| | Overall survival | | |
|---|---|---|---|
| Prognostic factor | HR | 95% C.I. | p-value |
| UICC grade | 2.105 | 1.800-2.410 | 0.015 |
| Ki-67 >=20 | 1.694 | 0.818-2.571 | 0.547 |
| Age < 40 | 0.306 | −0.232-0.844 | 0.028 |
| pSer 472 RelB nuclear labelling | 3.812 | 3.368-4.255 | 0.003 |
| (percentage of cells) | | | |
| HER2 negative | 0.246 | −0.560-1.053 | 0.082 |
| Mitotic index | 1.373 | 0.882-1.865 | 0.519 |
| Vascular emboli | 1.300 | 0.904-1.695 | 0.508 |
| TNBC status | 0.809 | 0.316-1.302 | 0.668 |
| Histological Grade (SBR) = 3 | 2.061 | 1.246-2.876 | 0.375 |

TABLE 9

| | DMFS | | |
|---|---|---|---|
| Prognostic factor | HR | 95% C.I. | p-value |
| UICC grade | 2.608 | 2.219-2.996 | 0.014 |
| Ki-67 >=20 | 3.231 | 2.049-4.412 | 0.321 |
| Age < 40 | 0.164 | −0.434-0.762 | 0.003 |
| pSer 472 RelB nuclear labelling | 6.439 | 5.897-6.980 | 0.001 |
| (percentage of cells) | | | |
| HER2 negative | 0.000 | −248.712-248.712 | 0.960 |
| Mitotic index | 0.863 | 0.272-1.454 | 0.803 |
| Vascular emboli | 1.552 | 0.981-2.122 | 0.441 |
| TNBC status | 1.245 | 0.652-1.839 | 0.711 |
| Histological Grade (SBR) = 3 | 2.565 | 1.498-3.631 | 0.377 |

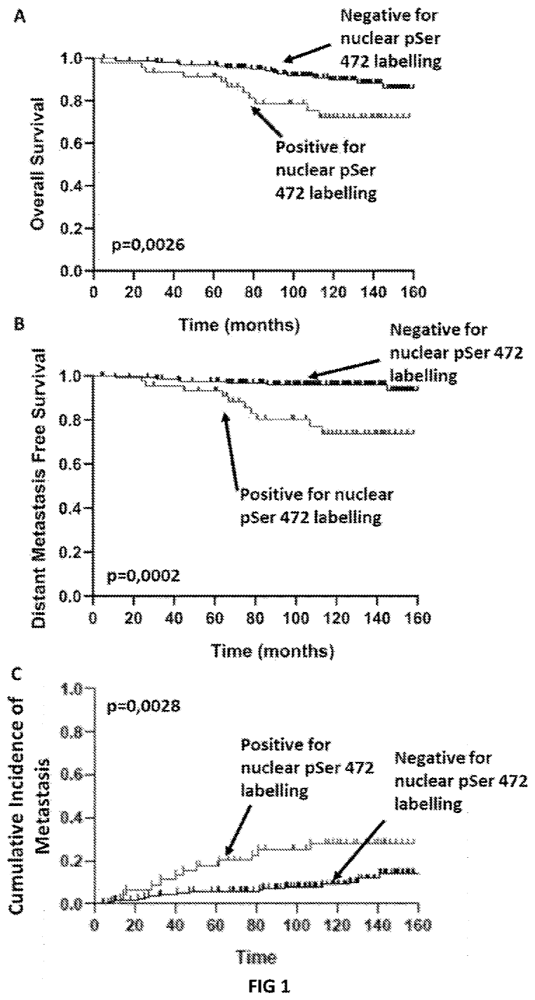

FIG. 1 (FIG. 1) depicts the cumulative incidence of metastases (FIG. 1C), Over Survival (FIG. 1A) and Distant Metastasis-Free Survival data (FIG. 1B) according to nuclear pSer472 RelB staining found in tumour sample from the subjects in a Kaplan-Meier plot. For all these three criteria, a significant difference is observed when patients are segregated as a function of the p5472-RelB nuclear labelling status found for their cancer cell sample (OS, p=0.0026; DMFS, p=0.0002; cumulative incidence of metastases p=0.0028).

Cytoplasmic pSer 472 RelB Labelling Features are Significantly Associated with Bad Prognostic Factors Used in Clinical Evaluation of Disease

TABLE 10

| | Diffuse cytoplasmic labelling | | | Discrete cytoplasmic labelling | | |
|---|---|---|---|---|---|---|
| | | % of | | | | % of |
| | | labelled | | Size of | Number | labelled |
| | Intensity | cells | score† | dots | of dots | cells |
| TNBC vs other | ns | ns | p = 0.01 | p = 0.001 | p = 0.001 | p = 0.001 |
| molecular types | | | | | | |
| Mitotic index | ns | p = 0.02 | ns | p < 0.001 | p < 0.005 | ns |
| Ki-67 | ns | p = 0.002 | p = 0.016 | p < 0.0001 | p < 0.0001 | p = 0.003 |
| UICC grade | ns | ns | ns | p = 0.003 | ns | ns |
| Presence of | ns | ns | ns | ns | ns | p = 0.004 |
| emboli | | | | | | |
| TILs | ns | p = 0.04 | ns | ns | ns | ns |

ns: not statistically associated;
†score is defined as in table 1

Statistical association has been searched for the 10 usual prognostic factors of cancer and phosphorylated Ser472 RelB cytoplasmic labelling features discovered by the inventors (table 10). Of note, 6 prognostic factors are found associated with features of cytoplasmic labelling for phosphorylated Ser472 RelB.

Cytoplasmic labelling, and more particularly discrete cytoplasmic labelling, is found associated with both mitotic index and Ki-67 index.

Then cytoplasmic labelling of phosphorylated Ser472 RelB could be considered as a prognostic factor especially in regard with cellular proliferation potential of cancer. Also, cytoplasmic labelling, especially discrete cytoplasmic labelling, is found significantly associated with TNBC molecular subtype of breast cancer.

Cytoplasmic pSer 472 RelB Labelling Features and Scoring

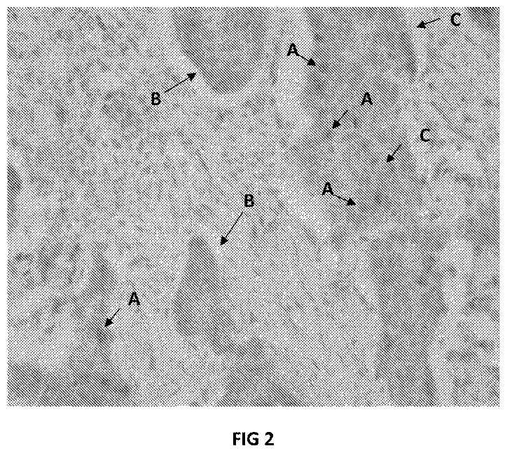

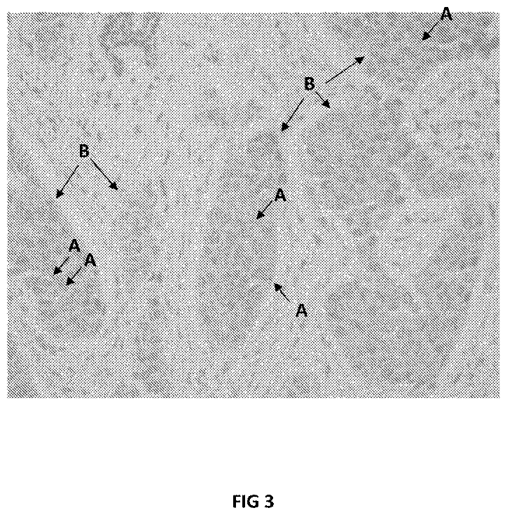

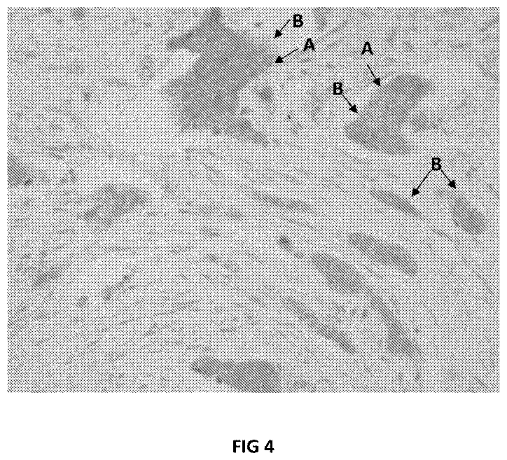

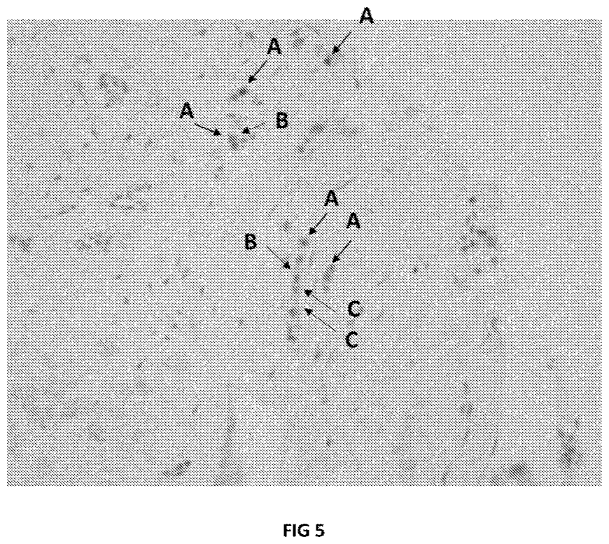

FIGS. 2-5 (FIG. 2-5) are representative results of IHC staining obtained with antibody against pS472 RelB (images were taken at 200× magnification), showing intensity criterion for diffuse cytoplasmic labelling ranging from 1 to 3, Cytoplasmic dots of a size ranging from 1 to 2 and presence or absence of nuclear labeling, for TNBC or luminal type A cancer breast sample. IHC staining with Antibody against pS472 RelB provide very clear and specific features which are easily usable in cancer histological examination.

### REFERENCES

Ahirwar R, Vellarikkal S K, Sett A, et al. Aptamer-Assisted Detection of the Altered Expression of Estrogen Receptor Alpha in Human Breast Cancer. PLoS One. 2016; 11(4):e0153001.
Ausubel et al. CURRENT PROTOCOLS IN MOLECULAR BIOLOGY, 1989.
Authier H, Billot K, Derudder E, et al. IKK phosphorylates RelB to modulate its promoter specificity and promote fibroblast migration downstream of TNF receptors. Proc Natl Acad Sci USA. 2014; 111(41):14794-14799.
Baud V, Collares D. Post-Translational Modifications of RelB NF-κB Subunit and Associated Functions. Cells. 2016;5(2):22.
Baud V, Karin M. Is NF-kappaB a good target for cancer therapy? Hopes and pitfalls. Nat Rev Drug Discov. 2009; 8(1):33-40.
Brady G, Jantzen H M, Bernard H U, Brown R, Schütz G, Hashimoto-Gotoh T. New cosmid vectors developed for eukaryotic DNA cloning. Gene. 1984; 27(2):223-232.
Chow S, Patel H, Hedley D W. Measurement of MAP kinase activation by flow cytometry using phospho-specific antibodies to MEK and ERK: potential for pharmacodynamic monitoring of signal transduction inhibitors. Cytometry. 2001; 46(2):72-78.
Conrad R C, Giver L, Tian Y, Ellington A D. In vitro selection of nucleic acid aptamers that bind proteins. Methods Enzymol. 1996; 267:336-367.
Czernik, Production of phosphorylation state-specific antibodies. Methods In Enzymology, 201: 264-283 (1991).
de Marco A. Recombinant expression of nanobodies and nanobody-derived immunoreagents. Protein Expr Purif. 2020; 172:105645.
Ellington A D, Szostak J W. In vitro selection of RNA molecules that bind specific ligands. Nature. 1990; 346(6287):818-822.
Hofmarcher T, Brklvik G, Svedman C, Lindgren P, Jönsson B and Wilking N.
Comparator Report on Cancer in Europe 2019—Disease Burden, Costs and Access to Medicines. IHE Report 2019:7, IHE: Lund, Sweden.
Huse W D, Sastry L, Iverson SA, et al. Generation of a large combinatorial library of the immunoglobulin repertoire in phage lambda. Science. 1989; 246(4935):1275-1281.
Köhler G, Milstein C. Derivation of specific antibody-producing tissue culture and tumor lines by cell fusion. Eur J Immunol. 1976; 6(7):511-519.
Köhler G, Milstein C. Continuous cultures of fused cells secreting antibody of predefined specificity. Nature. 1975; 256(5517):495-497.
Lefranc M P, Pommie C, Ruiz M, et al. IMGT unique numbering for immunoglobulin and T cell receptor variable domains and Ig superfamily V-like domains. Dev Comp Immunol. 2003; 27(1):55-77.
McCauley T G, Hamaguchi N, Stanton M. Aptamer-based biosensor arrays for detection and quantification of biological macromolecules. Anal Biochem. 2003; 319(2):244-250.
Mackillop, W. J. (2006). The Importance of Prognosis in Cancer Medicine. In TNM Online (eds L. H. Sobin, M. K. Gospodarowicz, B. O'Sullivan, L. H. Sobin, D. Henson and R. Hutter).
Maier H J, Marienfeld R, Wirth T, Baumann B (2003) Critical role of RelB serine 368 for dimerization and p100 stabilization. J Biol Chem 278: 39242-392500eckinghaus A, Ghosh S. The NF-kappaB family of transcription factors and its regulation. Cold Spring Harb Perspect Biol. 2009; 1(4):a000034.
Marienfeld R, May M J, Berberich I, Serfling E, Ghosh S, et al. (2003) RelB forms transcriptionally inactive complexes with RelA/p65. J Biol Chem 278: 19852-19860
Merrifield, J. Am. Chem. Soc. 1962
Miyaji H, Harada N, Mizukami T, Sato S, Fujiyoshi N, Itoh S. Efficient expression of human beta-interferon in Namalwa KJM-1 cells adapted to serum-free medium by a dhfr gene coamplification method. Cytotechnology. 1990; 4(2):173-180.
Mizukami T, Itoh S. A new SV40-based vector developed for cDNA expression in animal cells. J Biochem. 1987; 101(5):1307-1310.
Mullinax R L, Gross E A, Amberg JR, et al. Identification of human antibody fragment clones specific for tetanus toxoid in a bacteriophage lambda immunoexpression library. Proc Natl Acad Sci USA. 1990; 87(20):8095-8099.
NeedlemanSB and Wunsch C D. A general method applicable to the search for similarities in the amino acid sequence of two proteins J. Mol. Biol. 1970 March;48(3):443-53.
Neuberger M S, Williams G T, Fox R O. Recombinant antibodies possessing novel effector functions. Nature. 1984; 312(5995):604-608.
O'Hare K, Benoist C, Breathnach R. Transformation of mouse fibroblasts to methotrexate resistance by a recombinant plasmid expressing a prokaryotic dihydrofolate reductase. Proc Natl Acad Sci USA. 1981; 78(3):1527-1531.
Ramos-Vara J A, Miller M A. When tissue antigens and antibodies get along: revisiting the technical aspects of immunohistochemistry—the red, brown, and blue technique. Vet Pathol. 2014; 51(1):42-87.
Spira G, Bargellesi A, Teillaud J L, Scharff M D. The identification of monoclonal class switch variants by sib selection and an ELISA assay. J Immunol Methods. 1984; 74(2):307-315.
Steplewski Z, Spira G, Blaszczyk M, et al. Isolation and characterization of anti-monosialoganglioside monoclonal antibody 19-9 class-switch variants. Proc Natl Acad Sci U S A. 1985; 82(24):8653-8657.
Sun S C. The non-canonical NF-κB pathway in immunity and inflammation. Nat Rev Immunol. 2017 September;17(9):545-558.
Zamay G S, Ivanchenko T I, Zamay TN, et al. DNA Aptamers for the Characterization of Histological Structure of Lung Adenocarcinoma. Mol Ther Nucleic Acids. 2017; 6:150-162.

### Reference to a “Sequence Listing,” a Table, or a Computer Program Listing Appendix Submitted as an Ascii Text File

The material in the ASCII text file, name “APIC-68308-Sequence-Listing_ST25.txt”, created May 2, 2023, file size 81,920 bytes, is hereby incorporated by reference.

1. An in vitro method for classifying a subject afflicted with a solid cancer as having a good prognosis or a poor prognosis, comprising detecting in a cancer cell sample from the subject phosphorylated RelB protein of SEQ ID NO:1 at the serine residue 472 and/or detecting a RelB homolog phosphorylated at a corresponding serine.

2. The method according to claim 1, wherein the phosphorylated RelB protein of SEQ ID NO:1 at the serine residue 472 and/or the RelB homolog phosphorylated at a corresponding serine is detected using an antibody, or a fragment thereof.

3. The method according to claim 1, wherein detecting the phosphorylated RelB protein of SEQ ID NO:1 at the serine residue 472 and/or detecting the RelB homolog phosphorylated at a corresponding serine comprises detecting:
a cytoplasmic labelling, and/or
a nuclear labelling,
in cells of the cancer cell sample, the cytoplasmic or nuclear labelling corresponding to the phosphorylated RelB and/or the phosphorylated RelB homolog.

4. The method according to claim 3, wherein detecting the cytoplasmic labelling for the phosphorylated RelB protein of SEQ ID NO:1 at the serine residue 472 and/or the RelB homolog phosphorylated at a corresponding serine comprises:
when a diffuse labelling is detected, quantifying the intensity of the cytoplasmic labelling and/or the percentage of cells that are labelled and/or the percentage of the surface of a tissue biopsy sample that is labelled, or
when a discrete labelling with dots is observed, quantifying the size of the dots and/or quantifying their number either in cells or in the whole cancer cell sample and/or the percentage of cells of the cancer cell sample that are labelled by the dots and/or the percentage of the surface of the cancer cell sample that is labelled by the dots.

5. The method according to claim 3, wherein detecting the nuclear labelling for the phosphorylated RelB protein of SEQ ID NO:1 at the serine residue 472 and/or the RelB homolog phosphorylated at a corresponding serine comprises detecting a presence or an absence of the nuclear labelling and/or the percentage of cells of the cancer cell sample with the nuclear labelling and/or the percentage of the surface of a tissue biopsy sample that is labelled with the nuclear labelling.

6. The method according to claim 3, wherein detecting the nuclear labelling for the phosphorylated RelB protein of SEQ ID NO:1 at the serine residue 472 and/or the RelB homolog phosphorylated at a corresponding serine is indicative of a poor prognosis in the subject.

7. The method according to claim 6, wherein the poor prognosis comprises a significant increase of the risk for the occurrence of metastases for the subject and/or of a significant decrease of overall survival expectancy.

8. The method according to claim 3, wherein detecting a discrete labelling with dots in cytoplasm of the cancer cell sample is indicative of an increased risk of suffering from a cancer with a high proliferation potential and/or sensitivity to chemotherapy for the subject.

9. The method according to claim 1, wherein detecting phosphorylated RelB protein of SEQ ID NO:1 at the serine residue 472 and/or detecting the RelB homolog phosphorylated at a corresponding serine is performed using an antibody, a fragment thereof, or a single chain antibody comprising at least one CDR sequence selected from SEQ ID NO:6, SEQ ID NO:7, SEQ ID NO:8 SEQ ID NO:9, SEQ ID NO:10, SEQ ID NO:11 or a functionally-conservative variant thereof.

10. The method according to claim 1, wherein the subject is suffering from a lung cancer, breast cancer, prostate cancer, cervical cancer, pancreatic cancer, colon cancer, ovarian cancer; stomach cancer, oesophagus cancer, mouth cancer, tongue cancer, gum cancer, skin cancer, melanoma, basal cell carcinoma, Kaposi's sarcoma, muscle cancer, heart cancer, liver cancer, bronchial cancer, cartilage cancer, bone cancer, testis cancer, kidney cancer, endometrium cancer, uterus cancer, bladder cancer, spleen cancer, thymus cancer, thyroid cancer, brain cancer, neuron cancer, mesothelioma, gall bladder cancer, ocular cancer, cancer of the cornea, cancer of uvea, cancer of the choroids, cancer of the macula, vitreous humor cancer, joint cancer, a synovium cancer, or a glioblastoma, preferably said subject is suffering from a breast cancer.

11. The method according to claim 1, wherein the subject is a mammal, preferably a human.

12. The method according to claim 1, wherein the subject is free of metastasis.

13. The method according to claim 1, wherein the cancer cell sample is a sample of a tumour tissue or of a metastasis from the subject.

14. The method according to claim 1, wherein the subject is suffering from breast cancer and wherein detecting a discrete labelling with dots in cytoplasm of the cancer cell sample is indicative of an increased risk of suffering from a triple-negative breast cancer for the subject.

15. A kit comprising:
an antibody, or a fragment thereof, which binds the phosphorylated RelB protein of SEQ ID NO:1 at the serine residue 472 and/or a RelB homolog phosphorylated at a corresponding serine, and
an instruction to allow classification of the subject afflicted with cancer as having a good prognosis or a poor prognosis by detecting nuclear and/or cytoplasmic labelling with the antibody or fragment thereof.

16. The method according to claim 2, wherein the antibody or the fragment thereof comprises a monoclonal antibody, a fragment of the monoclonal antibody, or an aptamer.

17. The method according to claim 10, wherein the subject is suffering from a breast cancer.

18. The method according to claim 11, wherein the subject is a mammal.

19. The kit according to claim 15, wherein the antibody or the fragment thereof comprises a monoclonal antibody or a fragment of the monoclonal antibody.
